SEQUENCE LISTING (19 sequences)

SEQ ID NO: 1 - protein, 17 aa, synthetic construct
  REGION 1..17: note = Synthetic peptide
MYSFVSEETGTLIVNSV

SEQ ID NO: 2 - protein, 15 aa, synthetic construct
  REGION 1..15: note = Synthetic peptide
LLHAPATVCGPKKST

SEQ ID NO: 3 - protein, 15 aa, synthetic construct
  REGION 1..15: note = Synthetic peptide
NKKFLPFQQFGRDIA

SEQ ID NO: 4 - protein, 15 aa, synthetic construct
  REGION 1..15: note = Synthetic peptide
PHGVVFLHVTYVPAQ

SEQ ID NO: 5 - protein, 76 aa, unidentified
  REGION 1..76: note = SARS-CoV-2 envelope protein
MYSFVSEETGTLIVNSVLLFFAFVVFLLVTLAILTALRLCAYCCNIVNVSLVKPTVYVYSRVKNLNSSEGVPDLLV

SEQ ID NO: 6 - protein, 1297 aa, unidentified
  REGION 1..1297: note = SARS-CoV-2 spike protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQNVNCTEVPVAIHADQLTPTWRVYSTGSNVFQTRAGCLIGAEHVNNSYECDIPIGAGICASYQTQTNSPSRAGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGPALQIPFAMQMAYRFNGIGVTQNVLYENQKLIANQFNSAIGKIQDSLSSTPSALGKLQDVVNQNAQALNTLVKQLSSNFGAISSVLNDILSRLDKPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSLPETGGGSDYKDDDDKGGGGSGGGGSGGGGSGGGGSGGGGSHHHHHH

SEQ ID NO: 7 - protein, 8 aa, synthetic construct
  REGION 1..8: note = Synthetic peptide
MYSYVTEE

SEQ ID NO: 8 - protein, 7 aa, synthetic construct
  REGION 1..7: note = Synthetic peptide
YSFVTEE

SEQ ID NO: 9 - protein, 6 aa, synthetic construct
  REGION 1..6: note = Synthetic peptide
VSEETG

SEQ ID NO: 10 - protein, 11 aa, synthetic construct
  REGION 1..11: note = Synthetic peptide
YSFRSVREETG

SEQ ID NO: 11 - protein, 8 aa, synthetic construct
  REGION 1..8: note = Synthetic peptide
MYAFRSEE

SEQ ID NO: 12 - protein, 10 aa, synthetic construct
  REGION 1..10: note = Synthetic peptide
YSFFSEEGTG

SEQ ID NO: 13 - protein, 10 aa, synthetic construct
  REGION 1..10: note = Synthetic peptide
LHCPADVCGP

SEQ ID NO: 14 - protein, 9 aa, synthetic construct
  REGION 1..9: note = Synthetic peptide
LLHEHATVC

SEQ ID NO: 15 - protein, 16 aa, synthetic construct
  REGION 1..16: note = Synthetic peptide
LSEANTKFMFDLFQQF

SEQ ID NO: 16 - protein, 12 aa, synthetic construct
  REGION 1..12: note = Synthetic peptide
LTESNKSVLQFQ

SEQ ID NO: 17 - protein, 9 aa, synthetic construct
  REGION 1..9: note = Synthetic peptide
GVVFPHVSY

SEQ ID NO: 18 - protein, 11 aa, synthetic construct
  REGION 1..11: note = Synthetic peptide
FLYVHHTYVPA

SEQ ID NO: 19 - protein, 7 aa, synthetic construct
  REGION 1..7: note = Synthetic peptide
LNVTYVP

(19) United States
(12) Patent Application Publication
(10) Pub. No.: US 2025/0145667 A1
(43) Pub. Date: May 8, 2025

(54) PEPTIDE

(71) Applicants: QUEEN MARY UNIVERSITY OF LONDON, London (GB); BARTS HEALTH NHS TRUST, London (GB)

(72) Inventors: Federica Maria MARELLI-BERG, London (GB); Maria Paula LONGHI, London (GB); Saidi MOHIDDIN, London (GB)

(73) Assignees: QUEEN MARY UNIVERSITY OF LONDON, London (GB); BARTS HEALTH NHS TRUST, London (GB)

(21) Appl. No.: 18/715,522

(22) PCT Filed: Dec. 2, 2022
(86) PCT No.: PCT/GB2022/053079

§ 371 Date: May 31, 2024

(30) Foreign Application Priority Data
Dec. 2, 2021 (GB) 2117405.7

(51) Int. Cl. C07K 14/005; A61K 39/215; A61K 40/11; A61K 40/31; A61K 40/46; C07K 16/10; C12N 5/0783; G01N 33/569

(52) U.S. Cl. CPC C07K 14/005; A61K 39/215; A61K 40/11; A61K 40/31; A61K 40/46; C07K 16/1003; C12N 5/0637; G01N 33/56972; C12N 2510/00; C12N 2770/20022; G01N 2333/165

(57) ABSTRACT

The present invention relates to peptides derived from COVID-19 virus envelope protein and spike protein. The invention further relates to polynucleotides and vectors encoding said peptides, and antibodies and chimeric antigen receptors that bind to said peptides. The invention also relates to methods of diagnosis and prediction of conditions in a subject which leverage recognition of said peptides, such as by antigen-specific T cells.

### CROSS-REFERENCE TO RELATED APPLICATIONS

The present invention is filed under 35 U.S.C. § 371 as the U.S. national phase of International Application No. PCT/GB2022/053079, filed Dec. 2, 2022, which designated the U.S. and claims the right of priority of United Kingdom Patent Application No. 2117405.7, filed Dec. 2, 2021. The entire disclosures of the above-identified priority applications are hereby fully incorporated herein by reference.

### SEQUENCE LISTING

The instant application contains a Sequence Listing which has been submitted in ASCII format via EFS-Web and is hereby incorporated by reference in its entirety. Said ASCII copy, created on Dec. 2, 2022, is named 068901.002US_ST26 Sequence Listing and is 26 kilobytes in size.

### FIELD OF THE INVENTION

The present invention relates to peptides derived from COVID-19 virus envelope protein and spike protein. The invention further relates to polynucleotides and vectors encoding said peptides, and antibodies and chimeric antigen receptors that bind to said peptides. The invention also relates to methods of diagnosis and prediction of conditions in a subject which leverage recognition of said peptides, such as by antigen-specific T cells.

### BACKGROUND TO THE INVENTION

Severe acute respiratory syndrome-coronavirus-2 (SARS-CoV-2), the virus responsible for coronavirus disease 2019 (COVID-19) is a coronavirus with a single-stranded positive sense RNA genome of about 30,000 nucleotides in length. The virus genome encodes four structural proteins: the spike surface glycoprotein(S), small envelope protein (E), matrix protein (M) and nucleocapsid protein (N). The spike protein binds receptors on the host cell surface and mediates membrane fusion (Wrapp et al. Science 2020 367 (6483): 1260-1263). The envelope, matrix and nucleocapsid proteins function in encasing the RNA genome and mediating virus assembly, budding and envelope formation.

Infection with SARS-CoV-2 can cause, in addition to COVID-19, cardiac inflammation and damage (Kariyanna et al. Am J Med Case Rep 2020 8 (9): 299-305). Cardiac inflammation has also been reported after vaccination with mRNA-based COVID-19 vaccines, in particular the Moderna vaccine (mRNA-1273) and Pfizer/BioNTech vaccine (BNT162b2) (Albert et al. Rad Case Rep 2021 16 (8) 2142-2145; Bautista et al. Rev Esp Cardiol (Engl Ed) 2021 74 (9) 812-814; Diaz et al. JAMA 2021 326 (12): 1210-1212).

Cardiac inflammation enlarges and weakens the heart, reducing the efficiency with which blood is pumped around the body. Chronic cardiac inflammation can damage cardiomyocytes and in severe cases lead to disabling symptoms, progressive heart failure, cardiac arrest or stroke. The most common cause of cardiac inflammation is viral infection. There are three types of cardiac inflammation: myocarditis (inflammation of the heart muscle), pericarditis (inflammation of the outer lining of the heart) and endocarditis (inflammation of the inner lining of the heart). Myocarditis and pericarditis have been associated with both COVID-19 and vaccination against COVID-19. The COVID-19 virus may infect heart cells via interaction of it spike protein with angiotensin-converting enzyme 2 receptors (ACE2), directly causing cardiac damage and inflammation (Aleksova et al. In J Mol Sci 2021, 22, 4526). Alternatively or additionally, the COVID-19 virus may trigger a general hyperimmune response, thereby indirectly causing cardiac inflammation (Castiello et al. Heart Fail Rev 2021 1-11). Similarly, a COVID-19 vaccine may induce a hyperimmune response, leading to cardiac inflammation.

The incidence rate of myocarditis from infection with the COVID-19 virus has been reported as 11 events per 100,000 persons, whilst the risk of myocarditis from vaccination with mRNA-based COVID-19 vaccine is lower, at 2.7 events per 100,000 persons (Barda et al. N Engl J Med 2021 385:1078-90). Incidence rates of myocarditis post-vaccine are highest after a second dose of vaccine in young males between 16 and 19 years of age, and onset occurs within 21 days of first dose or 30 days of second dose (Mevorach et al. 2021 DOI: 10.1056/NEJMoa2109730).

The immune response to infection with SARS-CoV-2 and associated inflammatory conditions are mediated by effector T lymphocytes (effector T cells). Antigen-specific T cells are activated upon engagement of their T cell receptor (TCR) by a peptide antigen presented by a major histocompatibility complex (MHC) molecule on the surface of an antigen-presenting cell. The activated T cells then release cytokines which direct and regulate the immune response.

Effector T cells migrate to specific sites according to the set of adhesion molecules and chemokine receptors (“homing receptors”) they express. Migration of effector T cells to the heart (“cardiotropism”) is mediated by the hepatocyte growth factor (HGF) receptor cMet. Specifically, T cells primed in heart-draining lymph nodes in the presence of HGF produced in the cardiac parenchyma during inflammation are induced to express a molecular code that promotes their recirculation through the heart. In both mice and humans, cardiotropic T cells are characterized by expression of cMet and chemokine receptors CXCR3 and CCR4 (Komarowska et al. Immunity 2015 42, 1087-1099).

### SUMMARY OF THE INVENTION

The present inventors have identified that immunisation of animal subjects with peptides derived from COVID-19 virus spike protein and envelope protein which have similar sequences to parts of the sequences of cardiac proteins induces myocarditis and cardiac dysfunction. Without wishing to be bound by theory, it is hypothesised that COVID-19 virus spike protein and envelope protein in a subject suffering from COVID-19, or in a subject who has been vaccinated against COVID-19, induce an immune response which inadvertently also targets cardiac self-antigens, leading to cardiac inflammation. Accordingly, the peptides and agents which bind to the peptides (such as antibodies and CARs) may be useful in methods for analysing immune responses directed towards the COVID-19 virus, and in methods of diagnosis or prediction of COVID-19 or conditions associated with COVID-19, particularly inflammatory conditions (such as cardiac inflammatory conditions).

Generally, the invention concerns peptides derived from COVID-19 virus spike protein and envelope protein, related products such as polynucleotides and vectors encoding said peptides, and methods that utilise recognition of said peptides.

The invention provides a peptide selected from the group consisting of:

(SEQ ID NO 1)
MYSFVSEETGTLIVNSV,
(SEQ ID NO 2)
LLHAPATVCGPKKST,
(SEQ ID NO 3)
NKKFLPFQQFGRDIA,
(SEQ ID NO 4)
PHGVVFLHVTYVPAQ,

and variants thereof having at least 80% identity to any one of SEQ ID NOs 1, 2, 3 or 4.

The invention also provides a polynucleotide encoding one or more peptides of the invention.

The invention also provides a composition comprising one or more peptides of the invention and/or one or more polynucleotides of the invention.

The invention also provides a vector encoding one or more peptides of the invention, or a vector comprising one or more polynucleotides of the invention.

The invention also provides an antibody or chimeric antigen receptor (CAR) capable of binding to one or more peptides selected from the group consisting of SEQ ID NOs 1 to 4.

The invention also provides a CAR T cell expressing a CAR of the invention.

The invention also provides a pharmaceutical or immunomodulatory composition comprising:
one or more peptides of the invention; and/or
one or more polynucleotides of the invention; and/or
one more vectors of the invention; and/or
one or more antibodies or CARs of the invention; and/or
one or more CAR T cells of the invention;
   and a pharmaceutically acceptable carrier, vehicle, diluent or excipient.

The invention also provides a composition of the invention, a vector of the invention, an antibody or CAR of the invention, a CAR T cell of the invention or a pharmaceutical or immunomodulatory composition of the invention for use in inducing an immune response in a subject.

The invention also provides a method of inducing an immune response in a human or animal, said method comprising administering to the subject a composition of the invention, a vector of the invention, an antibody or CAR of the invention, a CAR T cell of the invention or a pharmaceutical or immunomodulatory composition of the invention.

The invention also provides an antibody or CAR of the invention, a CAR T cell of the invention, or a pharmaceutical or immunomodulatory composition of the invention comprising one or more antibodies or CARs of the invention and/or one or more CAR T cells of the invention, for use in the treatment or prevention of an inflammatory condition in a subject.

The invention also provides a method of treating or preventing an inflammatory condition in a subject, said method comprising administering to the subject an antibody or CAR of the invention, a CAR T cell of the invention, or a pharmaceutical or immunomodulatory composition of the invention comprising one or more antibodies or CARs of the invention and/or one or more CAR T cells of the invention.

The invention also provides an antibody or CAR of the invention, a CAR T cell of the invention, or a pharmaceutical or immunomodulatory composition of the invention comprising one or more antibodies or CARs of the invention and/or one or more CAR T cells of the invention, for use in treating COVID-19 in a subject.

The invention also provides a method of treating COVID-19 in a subject, said method comprising administering to the subject an antibody or CAR of the invention, a CAR T cell of the invention, or a pharmaceutical or immunomodulatory composition of the invention comprising one or more antibodies or CARs of the invention and/or one or more CAR T cells of the invention.

The invention also provides an antibody or CAR of the invention, a CAR Treg of the invention, or a pharmaceutical or immunomodulatory composition of the invention comprising one or more antibodies or CARs of the invention and/or one or more CAR Treg of the invention, for use in treating a cardiac inflammatory condition in a subject.

The invention also provides a method of treating a cardiac inflammatory condition in a subject, said method comprising administering to the subject an antibody or CAR of the invention, a CAR Treg of the invention, or a pharmaceutical or immunomodulatory composition of the invention comprising one or more antibodies or CARs of the invention and/or one or more CAR Tregs of the invention.

The invention also provides a major histocompatibility complex (MHC)-multimer comprising a peptide of the invention.

The invention also provides a kit comprising:
a composition of the invention; and/or
a vector of the invention; and/or
an antibody or CAR of the invention; and/or
a CAR T cell of the invention; and/or
a pharmaceutical or immunomodulatory composition of the invention; and/or
an MHC-multimer of the invention;
   and optionally instructions.

The invention also provides a method for determining in a sample a level of antigen-specific T cells expressing a T cell receptor (TCR) that binds to a peptide of the invention, the method comprising the steps of
(a) incubating the sample with one or more peptides of the invention; and
(b) determining the level of antigen-specific T cells in the sample by detecting binding of the one or more peptide to the TCR of antigen-specific T cells in the sample.

The invention also provides a method for diagnosing a condition in a subject, the method comprising the steps of:
(a) determining a level of antigen-specific T cells in a sample obtained from the subject using the method of the invention for determining in a sample a level of antigen-specific T cells; and
(b) comparing the level of antigen-specific T cells in the sample to an antigen-specific T cell reference value,
   wherein the level of antigen-specific T cells in the sample compared to the antigen-specific T cell reference value is indicative of the presence or absence of the condition in the subject.

The invention also provides a method for predicting whether a subject will develop a condition, the method comprising the steps of:
(a) determining a level of antigen-specific T cells in a sample obtained from the subject using the method of the invention for determining in a sample a level of antigen-specific T cells; and
(b) comparing the level of antigen-specific T cells in the sample to an antigen-specific T cell reference value,
   wherein the level of antigen-specific T cells in the sample compared to the antigen-specific T cell reference value is predictive of whether the subject will develop the condition.

The invention also provides a method of treating a subject suffering from a cardiac inflammatory condition, the method comprising the steps of:
(a) diagnosing a cardiac inflammatory condition using the method for diagnosing a cardiac inflammatory of the invention; and
(b) administering to the patient a therapeutically effective amount of a treatment, therapy or prophylaxis for a cardiac inflammatory condition.

The invention also provides a therapeutically effective amount of a treatment, therapy or prophylaxis for a cardiac inflammatory condition for use in a method of treating a subject suffering from a cardiac inflammatory condition, wherein the patient has been diagnosed with a cardiac inflammatory condition using the method for diagnosing a cardiac inflammatory condition of the invention.

### DESCRIPTION OF THE FIGURES

FIG. 1—Levels of circulating CD3+CD4+cMet+ T cells (A) and CD3+CD8+cMet+ T cells (B), heart weight to body weight ratio (C) and ejection fraction (E) in mice immunised subcutaneously with a cocktail of four COVID peptides (SEQ ID NOs 1-4) and the indicated adjuvant—none (control), alum+MPLA, R848, AS03+MPLA. Haematoxylin and eosin staining of a heart from a control mouse and a heart from a mouse immunised with peptide cocktail including AS03+MPLA adjuvant (D).

FIG. 2—Proliferation of CD4+cMet+ T cells upon immunisation of mice with peptide cocktail, with or without AS03+MPLA adjuvant (stimulated), or unstimulated.

FIG. 3—Levels of circulating CD4+cMet+ T cells and CD8+cMet+ T cells (A) and echocardiography data (B) from mice immunised intranasally with a cocktail of four COVID peptides (SEQ ID NOs 1-4) and the AS03+MPLA adjuvant (control mice immunised with AS03+MPLA adjuvant alone). Haematoxylin and eosin staining of hearts from two mice immunised with peptide cocktail including AS03+MPLA adjuvant (C).

FIG. 4—Levels of circulating CD3+CD4+cMet+ T cells and CD3+CD8+cMet+ T cells in mice immunised with individual COVID peptides in AS03+MPLA adjuvant (control mice immunised with AS03+MPLA adjuvant alone).

FIG. 5—Gating strategy from data for single cells (A) and lymphocytes identified within the blood (B). Granulocyte and monocyte populations also seen.

### DETAILED DESCRIPTION

### Peptides

The invention provides a peptide selected from the group consisting of:

(SEQ ID NO 1)
MYSFVSEETGTLIVNSV,
(SEQ ID NO 2)
LLHAPATVCGPKKST,
(SEQ ID NO 3)
NKKFLPFQQFGRDIA,
(SEQ ID NO 4)
PHGVVFLHVTYVPAQ,

and variants thereof having at least 80% identity to any one of SEQ ID NOs 1, 2, 3 or 4.

In some embodiments, the peptide is SEQ ID NO 1 or a variant thereof having at least 80% identity to SEQ ID NO 1. In some embodiments, the peptide is SEQ ID NO 2 or a variant thereof having at least 80% identity to SEQ ID NO 2. In some embodiments, the peptide is SEQ ID NO 3 or a variant thereof having at least 80% identity to SEQ ID NO 3. In some embodiments, the peptide is SEQ ID NO 4 or a variant thereof having at least 80% identity to SEQ ID NO 4. In some embodiments, the peptide is SEQ ID NO 1. In some embodiments, the peptide is SEQ ID NO 2. In some embodiments, the peptide is SEQ ID NO 3. In some embodiments, the peptide is SEQ ID NO 4. The peptide may be derived from COVID-19 virus spike protein. Accordingly, in some embodiments the peptide is selected from the group consisting of SEQ ID NOs 2, 3 and 4 and variants thereof having at least 80% identity to any one of SEQ ID NOs 2, 3 or 4.

The term “peptides of the invention” means any of SEQ ID NOs, 1, 2, 3 or 4 or any variant of any one of SEQ ID NOs 1, 2, 3 or 4 (wherein a variant has at least 80% identity to any one of SEQ ID NOs 1 to 4), or any subset of peptides within that group as defined herein. The term “peptide” is used herein to mean a series of residues, typically L-amino acids, connected one to the other, typically by peptide bonds between the amino group and carboxyl group on the α-carbon of adjacent amino acids. The term includes modified peptides and synthetic peptide analogues. The terms “peptide”, “polypeptide”, “protein” and “amino acid sequence” are used interchangeably herein. The peptides of the invention may be isolated.

Throughout the present description and claims the conventional three-letter and one-letter codes for naturally occurring amino acids are used, i.e. A (Ala), G (Giy), L (Leu), I (lie), V (Val), F (Phe), W (Trp), S (Ser), T (Thr), Y (Tyr), N (Asn), Q (Gin), D (Asp), E (Giu), K (Lys), R (Arg), H (His), M (Met), C (Cys) and P (Pro); as well as generally accepted three-letter codes for other a-amino acids, such as sarcosine (Sar), norleucine (Nie), a-aminoisobutyric acid (Aib), 2,3-diaminopropanoic acid (Dap), 2,4-diaminobutanoic acid (Dab) and 2,5-diaminopentanoic acid (ornithine; Orn). Such other α-amino acids may be shown in square brackets “[ ]” (e.g. “[Aib]”) when used in a general formula or sequence in the present specification, especially when the rest of the formula or sequence is shown using the single letter code. Unless otherwise specified, amino acid residues in peptides of the invention are of the L-configuration. However, D-configuration amino acids may be incorporated. In the

present context, an amino acid code written with a small letter represents the D-configuration of said amino acid, e.g. “k” represents the D-configuration of lysine (K).

The peptide of the invention may be made using chemical methods (Peptide Chemistry, A practical Textbook. Mikos Bodansky, Springer-Verlag, Berlin.). For example, peptides can be synthesized by solid phase techniques (Roberge J Y et al. Science 1995 269:202-204), cleaved from the resin, and purified by preparative high performance liquid chromatography (e.g. Creighton Proteins Structures And Molecular Principles 1983 W H Freeman and Co, New York NY). Automated synthesis may be achieved, for example, using the ABI 43 1 A Peptide Synthesizer (Perkin Elmer) in accordance with the instructions provided by the manufacturer.

The peptide may alternatively be made by recombinant means, or by cleavage from a longer polypeptide. For example, the peptide may be obtained by cleavage from the S-antigen protein, which may be followed by modification of one or both ends. The composition of a peptide may be confirmed by amino acid analysis or sequencing (e.g. the Edman degradation procedure).

A “variant” peptide of the invention is a peptide consisting of an amino acid sequence that has at least 80% identity to the amino acid sequence of any one of SEQ ID NOs 1, 2, 3 or 4.

In some embodiments, the peptide of the invention has at least 80%, 81%, 82%, 83%, 84%, 85%, 86%, 87%, 88%, 89%, 90%, 91%, 92%, 93%, 94%, 95%, 96%, 97%, 98%, 99% or 100% identity to any one of SEQ ID NOs 1, 2, 3 or 4. In some embodiments, the peptide has at least 80%, 85%, 90%, 95%, or 100% identity to any one of SEQ ID NOs 1, 2, 3 or 4.

In some embodiments, the peptide of the invention has at least 80%, 81%, 82%, 83%, 84%, 85%, 86%, 87%, 88%, 89%, 90%, 91%, 92%, 93%, 94%, 95%, 96%, 97%, 98%, 99% or 100% identity to SEQ ID NO 1. In some embodiments, the peptide of the invention has at least 80%, 81%, 82%, 83%, 84%, 85%, 86%, 87%, 88%, 89%, 90%, 91%, 92%, 93%, 94%, 95%, 96%, 97%, 98%, 99% or 100% identity to SEQ ID NO 2. In some embodiments, the peptide of the invention has at least 80%, 81%, 82%, 83%, 84%, 85%, 86%, 87%, 88%, 89%, 90%, 91%, 92%, 93%, 94%, 95%, 96%, 97%, 98%, 99% or 100% identity to SEQ ID NO 3. In some embodiments, the peptide of the invention has at least 80%, 81%, 82%, 83%, 84%, 85%, 86%, 87%, 88%, 89%, 90%, 91%, 92%, 93%, 94%, 95%, 96%, 97%, 98%, 99% or 100% identity to SEQ ID NO 4.

The degree of identity (expressed as a percentage) of the sequence of a given variant peptide with the sequence of SEQ ID NO 1, 2, 3 or 4 is defined as the percentage of amino acid residues in the variant sequence that are identical to the amino acid residues in the sequence of SEQ ID NO 1, 2, 3 or 4, after aligning the sequences and introducing gaps, if necessary, to achieve the maximum percentage sequence identity, and not considering any conservative substitutions as part of the sequence identity. Sequence alignment can be carried out by the skilled person using techniques well known in the art, for example using publicly available software. Examples of such software include Clustal W (Thompson et al. Nucleic Acids Res. 1994 22:4673-4680), ALIGN (Myers et al. CABIOS 1988 4:1-17), FASTA (Pearson et al. PNAS 1988 85:2444-2448; Pearson Methods Enzymol. 1990 183:63-98), BLAST and BLAST2 (Altschul et al. Nucleic Acids Res. 1997 25:3389-3402) and the Dali server at the European Bioinformatics Institute (Holm J. Mol. Biol. 1993 233:123-38; Holm Trends Biochem. Sci. 1995 20:478-480; Holm Nucleic Acid Res. 1998 26:316-9).

The percentage sequence identities used in the context of the present invention may be determined using these programs with their default settings. More generally, the skilled worker can readily determine appropriate parameters for determining alignment, including any algorithms needed to achieve maximal alignment over the full length of the sequences being compared.

It will be appreciated that, according to the definition of a variant peptide as having at least 80% identity with any one of SEQ ID NOs 1 to 4, a variant peptide of the invention may be longer or shorter than any of SEQ ID NOs 1, 2, 3 or 4, and the maximum or minimum length of a variant peptide of the invention depends upon with which of SEQ ID NOs 1, 2, 3 or 4 the variant peptide has identity.

### Polynucleotides

The invention provides a polynucleotide encoding one or more peptides of the invention. In other words, the invention provides a polynucleotide encoding a peptide selected from the group consisting of:

(SEQ ID NO 1)
MYSFVSEETGTLIVNSV,
(SEQ ID NO 2)
LLHAPATVCGPKKST,
(SEQ ID NO 3)
NKKFLPFQQFGRDIA,
(SEQ ID NO 4)
PHGVVFLHVTYVPAQ,

and variants thereof having at least 80% identity to any one of SEQ ID NOs 1 to 4.

In some embodiments, the polynucleotide encodes SEQ ID NO 1 or a variant thereof having at least 80% identity to SEQ ID NO 1. In some embodiments, the polynucleotide encodes SEQ ID NO 2 or a variant thereof having at least 80% identity to SEQ ID NO 2. In some embodiments, the polynucleotide encodes SEQ ID NO 3 or a variant thereof having at least 80% identity to SEQ ID NO 3. In some embodiments, the polynucleotide encodes SEQ ID NO 4 or a variant thereof having at least 80% identity to SEQ ID NO 4. In some embodiments, the polynucleotide encodes SEQ ID NO 1. In some embodiments, the polynucleotide encodes SEQ ID NO 2. In some embodiments, the polynucleotide encodes SEQ ID NO 3. In some embodiments, the polynucleotide encodes SEQ ID NO 4. The polynucleotide may encode a peptide derived from COVID-19 virus spike protein. Accordingly, in some embodiments the polynucleotide encodes a peptide selected from the group consisting of SEQ ID NOs 2, 3 and 4 and variants thereof having at least 80% identity to any one of SEQ ID NOs 2, 3 or 4.

The term “polynucleotides of the invention” means any polynucleotide encoding one or more peptides of the invention (i.e. SEQ ID NO 1, 2, 3 or 4, or any variants of any of SEQ ID NO 1, 2, 3 or 4). The terms “polynucleotide”, “nucleotide sequence”, “nucleic acid” and “nucleic acid sequence” are used interchangeably herein to mean a chain of covalently bonded nucleotide monomers, as known in the art. The polynucleotide may be of genomic, synthetic or recombinant origin, may be double-stranded or single-stranded, and may be a sense strand, an anti-sense strand or both. The polynucleotide may be DNA (such as genomic DNA, cDNA or synthetic DNA), RNA (such as mRNA or synthetic RNA) or a mixture of DNA and RNA. In some embodiments, the polynucleotide is cDNA sequence encoding one or more peptides of the present invention.

In some embodiments, the polynucleotide of the present invention does not include the native nucleotide sequence when in its natural environment and when it is linked to its naturally associated sequence(s) that is/are also in its/their natural environment. For ease of reference, herein these embodiments are called the “non-native polynucleotide”. In this regard, the term “native polynucleotide” means an entire nucleotide sequence that is in its native environment and when operatively linked to an entire promoter with which it is naturally associated, which promoter is also in its native environment. However, the peptides as described herein can be isolated and/or purified post expression from a polynucleotide in its native organism. However, the peptides as described herein may be expressed from a polynucleotide in its native organism but wherein the polynucleotide is not under the control of the promoter with which it is naturally associated within that organism.

The polynucleotide of the present invention is typically synthesised using standard chemical methods well known in the art (see for example Caruthers et al. 1980 Nuc Acids Res Symp Ser 215-23 and Horn et al. 1980 Nuc Acids Res Symp Ser 225-232). For example, in the phosphoroamidite method (Beucage et al. 1981 Tetrahedron Letters 22 1859-1869, Matthes et al., 1984 EMBO J. 3, 801-805), oligonucleotides are synthesised (e.g. in an automatic DNA synthesiser), purified, annealed, ligated and cloned in appropriate vectors.

The polynucleotide may alternatively be prepared by any other appropriate technique known in the art, such as recombinant DNA techniques, isolation and/or purification from a cell or organism, by polymerase chain reaction (PCR) using specific primers (see for example U.S. Pat. No. 4,683,202 and Saiki et al. 1988 Science 239, 487-491) or from a genomic DNA and/or cDNA library from an organism producing a peptide of the invention.

The term “encoding” means that a sequence of nucleotides in the polynucleotide when read according to the genetic code corresponds to the amino acid sequence of one or more of the peptides of the invention (i.e. one or more of SEQ ID NOs 1, 2, 3 or 4 or a variant of any of SEQ ID NOs 1, 2, 3 or 4). In other words, when all or part of the polynucleotide is translated, one or more of the peptides of the invention is produced. Put another way, “encoding” means that comprised in the polynucleotide is a series of nucleotide triplets that are codons in an order corresponding to the amino acid sequence of one or more peptides of the invention. The genetic code and principles and mechanisms of translation of polynucleotides to produce polypeptide sequences are well known in the art. It will be appreciated that a polynucleotide that is DNA may first be transcribed to RNA, which in turn is translated to peptide.

The polynucleotide can be any nucleotide sequence that encodes a peptide of the invention. Thus, it will be appreciated that the polynucleotide of the invention encompasses a large variety of nucleotide sequences because the precise nucleotide sequence depends upon the sequence of the peptide the polynucleotide encodes, which itself is selected from a variety of sequences (SEQ ID NO 1, 2, 3, 4 or a variant of any of those). The large variety of sequences encompassed by the polynucleotide is due also to the known redundancy of the genetic code, whereby multiple different codons may code for the same amino acid.

The polynucleotide may comprise sequences in addition to the precise coding sequence of the peptides of the invention (i.e. sequences in addition to codons corresponding to the amino acid sequence of one or more peptides of the invention). These additional sequences may be further functional sequences, such as a promoter sequence, enhancer sequence, terminator sequence, operator sequence, start codon or stop codon. The additional sequences may be nonsense sequences, and may act as spacers between coding sequences.

In some embodiments, the polynucleotide encodes one peptide of the invention (i.e. one of SEQ ID NOs 1, 2, 3 or 4, or a variant of any of SEQ ID NOs 1, 2, 3 or 4). In some embodiments, the polynucleotide encodes more than one peptide of the invention. In some embodiments, the polynucleotide encodes two peptides of the invention. In some embodiments, the polynucleotide encodes three peptides of the invention. In some embodiments, the polynucleotide encodes four peptides of the invention. In some embodiments, the polynucleotide encodes one of SEQ ID NO 1, SEQ ID NO 2, SEQ ID NO 3 or SEQ ID NO 4. In some embodiments, the polynucleotide encodes SEQ ID NOs 1, 2, 3 and 4. In embodiments wherein the polynucleotide encodes more than one peptide of the invention, the coding sequences for the peptides may be separated by other sequences, such as nonsense sequences or promoter sequences. In such embodiments, the peptide coding sequences may be under control of the same promoter or under control of different promoters.

### Composition

The invention provides a composition comprising one or more peptides of the invention and/or one or more polynucleotides of the invention.

In some embodiments, the composition comprises one peptide of the invention (i.e. one of SEQ ID NOs 1, 2, 3 or 4, or a variant having at least 80% identity to any one of SEQ ID NOS 1, 2, 3 or 4). In some embodiments, the composition comprises more than one peptide of the invention. In some embodiments, the composition comprises two peptides of the invention. In some embodiments, the composition comprises three peptides of the invention. In some embodiments, the composition comprises four peptides of the invention. In some embodiments, the composition comprises SEQ ID NO 1 or a variant thereof having at least 80% identity to SEQ ID NO 1. In some embodiments, the composition comprises SEQ ID NO 2 or a variant thereof having at least 80% identity to SEQ ID NO 2. In some embodiments, the composition comprises SEQ ID NO 3 or a variant thereof having at least 80% identity to SEQ ID NO 3. In some embodiments, the composition comprises SEQ ID NO 4 or a variant thereof having at least 80% identity to SEQ ID NO 4. The peptide may be derived from COVID-19 virus spike protein. Accordingly, in some embodiments the composition comprises a peptide selected from the group consisting of SEQ ID NOs 2, 3 and 4 and variants thereof having at least 80% identity to any one of SEQ ID NOs 2, 3 or 4. In some embodiments, the composition comprises one of SEQ ID NO 1, SEQ ID NO 2, SEQ ID NO 3 or SEQ ID NO 4. In some embodiments, the composition comprises SEQ ID NOs 1, 2, 3 and 4. In some embodiments, the composition comprises SEQ ID NOs 2, 3 and 4.

In some embodiments, the composition comprises one polynucleotide of the invention. In some embodiments, the composition comprises a polynucleotide encoding SEQ ID NOs 1, 2, 3 and 4. In some embodiments, the composition comprises more than one polynucleotide of the invention. In some embodiments, the composition comprises two polynucleotides of the invention. In some embodiments, the composition comprises three polynucleotides of the invention. In some embodiments, the composition comprises four polynucleotides of the invention. In embodiments wherein the composition comprises more than one polynucleotide of the invention, each polynucleotide of the invention may encode a different peptide of the invention. In some such embodiments, the composition comprises a polynucleotide encoding SEQ ID NO 1, a polynucleotide encoding SEQ ID NO 2, a polynucleotide encoding SEQ ID NO 3 and a polynucleotide encoding SEQ ID NO 4. In embodiments wherein the composition comprises more than one polynucleotide of the invention, one or more of the polynucleotides may encode a single peptide of the invention and one or more other polynucleotides may encode more than one peptide of the invention.

In some embodiments, the composition comprises one or more peptides of the invention and one or more polynucleotides of the invention. In other words, in some embodiments, the composition comprises a mixture of peptides of the invention and polynucleotides of the invention. That is, in such embodiments, one or more peptides of the invention per se are present in the composition alongside one or more polynucleotides encoding one or more peptides of the invention.

### Adjuvant

In some embodiments, the composition of the invention further comprises an adjuvant. The term “adjuvant” means a substance which increases the pharmacological potency of another substance. In the context of the present invention, the adjuvant is a substance which can enhance the immune response of a subject. In some embodiments, the adjuvant enhances the immune response of a subject to one or more peptides of the invention.

In some embodiments, the adjuvant is selected from poly-IC, complete Freund's adjuvant, cytosine-phosphate-guanine (CpG), alum+LPS derivative, R848 and AS03+LPS derivative. In some embodiments, the adjuvant is selected from alum, R848 and AS03+LPS derivative. In some embodiments, the LPS derivative is MPLA. In other words, in some embodiments, the adjuvant is AS03+MPLA.

Poly-IC (polyinosine-polycytidine) is a synthetic double-stranded RNA molecule that elicits an immune response by mimicking a viral infection. Poly-IC is commercially available (for example Invivogen cat. no. tlrl-pic).

Complete Freund's adjuvant is a solution of inactivated mycobacteria, and is commercially available (for example Santa Cruz Biotechnology sc-3727).

CpG is a single-stranded synthetic DNA molecule comprising cytosine followed by guanine (CpG) motifs. CpG is commercially available (for example Invivogen ODN 1585).

“Alum” refers to aluminium salts such as aluminium hydroxide, aluminium phosphate and aluminium potassium sulphate. Aluminium salts are readily commercially available (for example, Invivogen Alhydrogel (aluminium hydroxide gel), cat. no. vac-alu-250).

R848 (also known as resiquimod) is a TLR7 and TLR8 agonist small molecule with the following structural formula.

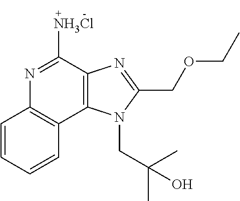

R848 is readily available commercially (for example, Miltenyi Biotec 130-109-376).

In preferred embodiments, the adjuvant is AS03+LPS derivative. In other words, in some embodiments, the adjuvant is a combination of AS03 and an LPS derivative. AS03 stands for “Adjuvant System 03”, and consists of α-tocopherol, squalene and polysorbate 80 in an oil-in-water emulsion (Garçon et al. 2012 Expert Rev Vaccines 11 (3) 349-366). LPS stands for lipopolysaccharide, which is the major component of the outer membrane of Gram-negative bacteria.

In preferred embodiments, the LPS derivative is MPLA. MPLA (monophosphoryl lipid A) is a TLR4 agonist and commercially available (for example Merck cat. no. 699800P). In other embodiments the LPS derivative is glucopyranosyl lipid A (GLA).

### Vector

The invention provides a vector encoding one or more peptides of the invention, or a vector comprising one or more polynucleotides of the invention.

As is well known in the art, a vector is a tool that allows or facilitates the transfer of an entity from one environment to another. Vectors used in recombinant DNA techniques allow entities, such as polynucleotides of the invention, to be transferred into a host and/or a target cell for the purpose of replicating and/or expressing said polynucleotides. Thus, in some embodiments, the polynucleotides encoding the peptides of the invention may be incorporated into a recombinant replicable vector. The vector may be used to replicate the polynucleotide in a host cell. The vector may be used to express the polynucleotide in a host cell.

The vector may comprise elements that facilitate selection and replication of the vector. Accordingly, the vector may comprise an origin of replication. The vector may comprise one or more selectable markers genes, such as an ampicillin resistance gene in the case of a bacterial plasmid or a neomycin resistance gene for a mammalian vector.

In some embodiments, the vector is a transformation vector. The term “transformation vector” means a construct capable of being transferred from one species to another.

In some embodiments, the vector is an expression vector. The term “expression vector” means a vector capable of in vivo or in vitro or ex vivo expression of a coding sequence comprised in the vector, such as a coding sequence encoding the peptides of the invention, such as a polynucleotide of the invention. Accordingly, in addition to coding sequence(s), an expression vector comprises control sequences operably linked to the coding sequence(s), which effect expression of the coding sequence(s). The term “operably linked” refers to juxtaposition wherein the elements are in an arrangement allowing them to be functionally related (for example, a promoter is operably linked to a coding sequence if it controls the transcription of the coding sequence). Such control sequences include promoters (to effect transcription), optional operator sequences (to control such transcription) such as enhancer sequences or suppressor sequences, sequences encoding suitable mRNA ribosome binding sites, and sequences which control termination of transcription and translation, such as a polyadenylation signal. These control sequences may be selected to be compatible with a host cell in which the expression vector is designed to be used. The control sequences may be modified, for example by the addition of further transcriptional regulatory elements to make the level of transcription directed by the control sequences more responsive to transcriptional modulators.

Thus, in some embodiments, the vector comprises a polynucleotide of the invention operably linked to control sequences that provide for the expression of the polynucleotide by a host cell. In some embodiments, the polynucleotide of the invention is operably linked to a transcription unit. The term “transcription unit” refers to nucleic acid sequence containing coding sequences and signals for achieving expression of those coding sequences independently of any other coding sequences. Thus, each transcription unit generally comprises at least a promoter, an optional enhancer and a polyadenylation signal.

The term “promoter” means an RNA polymerase binding site. The term encompasses nucleic acid regions ranging in size and complexity from minimal promoters to promoters including upstream elements and enhancers. A promoter is typically selected from promoters which are functional in mammalian cells, although prokaryotic promoters and promoters functional in other eukaryotic cells may be used. A promoter is typically derived from promoter sequences of viral or eukaryotic genes. For example, it may be a promoter derived from the genome of a cell in which expression is to occur. With respect to eukaryotic promoters, they may be promoters that function in a ubiquitous manner (such as promoters of α-actin, β-actin or tubulin) or a tissue-specific manner (such as promoters of the genes for pyruvate kinase). A promoter may respond to specific stimuli, such as binding steroid hormone receptors. Viral promoters may also be used, for example the Moloney murine leukaemia virus long terminal repeat (MMLV LTR) promoter, the rous sarcoma virus (RSV) LTR promoter or the human cytomegalovirus (CMV) IE promoter. It may also be advantageous for a promoter to be inducible so that the levels of peptide can be regulated. “Inducible” means that the levels of expression obtained using the promoter can be regulated.

In addition, any of these promoters may be modified by the addition of further regulatory sequences, for example enhancer sequences. Chimeric promoters may also be used comprising sequence elements from two or more different promoters described above. The term “enhancer” includes a nucleic acid sequence which binds to other protein components of the transcription initiation complex and thus facilitates the initiation of transcription directed by its associated promoter.

Once transformed into a suitable host, a vector of the invention may replicate and function independently of the host genome or may integrate into the genome itself.

Expression of the peptides of the invention from the expression vector may be constitutive, such that the peptides are continually produced, or inducible, requiring a stimulus to initiate expression. In the case of inducible expression, peptide production can be initiated when required by, for example, addition of an inducer substance, such as dexamethasone or IPTG, to culture medium. Additionally, peptides produced from a vector of the invention by a host cell may be secreted or may be contained intracellularly depending on the sequence and/or the vector used. As will be understood by those of skill in the art, expression vectors containing the polypeptide coding sequences can be designed with signal sequences which direct secretion of the polypeptide coding sequences through a particular prokaryotic or eukaryotic cell membrane.

In some embodiments, the vector is a plasmid, which is the most commonly used form of expression vector. However, the invention encompasses other expression vectors that serve equivalent functions and which are, or become, known in the art. In some embodiments, the vector is a chromosome or artificial chromosome. In some embodiments, the vector is a phage particle. In some embodiments, the vector is a genomic insert.

The vectors of the invention may be introduced into host cells directly as naked nucleic acid constructs. The term “naked nucleic acid construct” refers to a plasmid comprising a polynucleotide sequence encoding a peptide of the invention together with a short promoter region to control its production. Such a construct is referred to as “naked” because the plasmids are not carried in a delivery vehicle. When such a plasmid enters a host cell, such as a eukaryotic cell, the peptides it encodes (such as the peptides of the invention) are transcribed and translated within the cell. In some embodiments, the vector comprises flanking sequences homologous to the host cell genome.

Alternatively, the vectors of the invention may be introduced into host cells using a variety of non-viral techniques known in the art, such as transfection, transformation, electroporation and biolistic transformation. The term “transfection” refers to a process using a non-viral vector to deliver a gene to a target mammalian cell. Typical transfection methods include electroporation, DNA biolistics, lipid-mediated transfection, compacted DNA-mediated transfection, liposomes, immunoliposomes, lipofectin, cationic agent-mediated (for example calcium phosphate and DEAE-dextran), cationic facial amphiphiles (CFAs) (Nature Biotechnology 1996 14 556), multivalent cations such as spermine, cationic lipids or polylysine, 1, 2,-bis (oleoyloxy)-3-(trimethylammonio) propane (DOTAP)-cholesterol complexes (Wolff and Trubetskoy 1998 Nature Biotechnology 16 421) lipofectants (for example Lipofectam™ and Transfectam™) and combinations thereof.

### Viral Vector

In some embodiments, the vector is a virus (i.e. a viral vector). In other words, the vectors comprising the polynucleotide of the invention may be introduced into suitable host cells using a variety of viral techniques known in the art, such as infection with recombinant viral vectors such as retroviruses, herpes simplex viruses and adenoviruses. In such embodiments, gene delivery is mediated by viral infection of a target cell.

In some embodiments, the vector is a recombinant viral vector. Suitable recombinant viral vectors include but are not limited to adenovirus vectors, adeno-associated viral (AAV) vectors, herpes-virus vectors, retroviral vectors, lentiviral vectors, baculoviral vectors, pox viral vectors or parvovirus vectors (see Kestler et al. 1999 Human Gene Ther 10 (10): 1619-32).

In some embodiments, the vector is a recombinant retroviral vector (RRV). Examples of retroviruses include: murine leukemia virus (MLV), human immunodeficiency virus (HIV), mouse mammary tumour virus (MMTV), Rous sarcoma virus (RSV), Fujinami sarcoma virus (FuSV), Moloney murine leukemia virus (Mo-MLV), FBR murine osteosarcoma virus (FBR MSV), Moloney murine sarcoma virus (Mo-MSV), Abelson murine leukemia virus (A-MLV), Avian myelocytomatosis virus-29 (MC29), and Avian erythroblastosis virus (AEV). A detailed list of retroviruses may also be found in Coffin et al (“Retroviruses” 1997 Cold Spring Harbour Laboratory Press Eds: J M Coffin, SM Hughes, HE Varmus pp 758-763). The term “recombinant retroviral vector” (RRV) refers to a vector with sufficient retroviral genetic information to allow packaging of an RNA genome, in the presence of packaging components, into a viral particle capable of infecting a target cell. Infection of the target cell includes reverse transcription and integration into the target cell genome. The RRV carries non-viral coding sequences which are to be delivered by the vector to the target cell. An RRV is incapable of independent replication to produce infectious retroviral particles within the final target cell. Usually the RRV lacks a functional gag-pol and/or env gene and/or other genes essential for replication. The vector of the present invention may be configured as a split-intron vector. A split intron vector is described in PCT patent application WO 99/15683.

In some embodiments, the vector is a lentiviral vector, which is a type of recombinant retroviral vector. Lentiviruses can be divided into primate and non-primate groups. Examples of primate lentiviruses include: the human immunodeficiency virus (HIV), the causative agent of human auto-immunodeficiency syndrome (AIDS), and the simian immunodeficiency virus (SIV). The non-primate lentiviral group includes the prototype “slow virus” visna/maedivirus (VMV), as well as the related caprine arthritis-encephalitis virus (CAEV), equine infectious anaemia virus (EIAV) and the more recently described feline immunodeficiency virus (FIV) and bovine immunodeficiency virus (BIV). A distinction between the lentivirus family and other types of retroviruses is that lentiviruses have the capability to infect both dividing and non-dividing cells (Lewis et al. 1992 EMBO. J 11:3053-3058; Lewis and Emerman 1994 J. Virol. 68:510-516). In contrast, other retroviruses are unable to infect non-dividing cells such as those that make up, for example, muscle, brain, lung and liver tissue.

In some embodiments, the vector is an adenoviral vector.

In some embodiments, the vector is a recombinant pox viral vector such as fowl pox virus (FPV), entomopox virus, vaccinia virus such as NYVAC, canarypox virus, MVA or other non-replicating viral vector systems such as those described for example in WO 95/30018.

### Antibody and CAR

The invention provides an antibody or chimeric antigen receptor (CAR) capable of binding to one or more peptides selected from the group consisting of SEQ ID NOs 1 to 4.

The term “antibody” refers to the well-known class of proteins used by the immune system to recognise and bind to specific antigens, such as pathogens. The antibody of the invention may be a polyclonal antibody, a monoclonal antibody, a chimeric antibody, a single chain antibody, or an antibody fragment, such as an Fab fragment, a fragment produced by an Fab expression library, an Fv fragment, an F(ab′) fragment, an F(ab′)2 fragment or a single chain Fv (scFv) fragment. The term “antibody” also include mimetics of any of the types of antibody and antibody fragments listed above, and fusion proteins and other synthetic proteins which comprise the antigen-binding site of the antibody. The antibodies and fragments thereof may be humanised antibodies. Neutralising antibodies, i.e. those which inhibit biological activity of the antigen, are especially preferred for diagnostics and therapeutics.

Antibodies may be produced by standard techniques, such as by immunisation with a peptide of the invention or by using a phage display library.

If polyclonal antibodies are desired, a selected mammal (e.g. mouse, rabbit, goat, horse, llama, etc.) and/or avian is immunised with a peptide of the invention (or a sequence comprising an immunological epitope thereof). Depending on the host species, various adjuvants may be used to increase immunological response. Serum from the immunised animal is collected and treated according to known procedures. If serum containing polyclonal antibodies to a peptide of the invention (or a sequence comprising an immunological epitope thereof) contains antibodies to other antigens, the polyclonal antibodies can be purified by immunoaffinity chromatography. Techniques for producing and processing polyclonal antisera are known in the art. In order that such antibodies may be made, the invention also provides polypeptides of the invention or fragments thereof haptenised to another polypeptide for use as immunogens in animals or humans.

Monoclonal antibodies directed against peptides of the invention (or a sequence comprising an immunological epitope thereof) can be readily produced by one skilled in the art and include the hybridoma technique (Koehler and Milstein 1975 Nature 256:495-497), the human B-cell hybridoma technique (Kosbor et al. 1983 Immunol Today 4:72; Cote et al., 1983 Proc Natl Acad Sci 80:2026-2030) and the EBV-hybridoma technique (Cole et al., 1985 Monoclonal Antibodies and Cancer Therapy, Alan Rickman Liss Inc. 77-96).

In addition, techniques developed for the production of “chimeric antibodies” (the splicing of mouse antibody genes to human antibody genes to obtain a molecule with appropriate antigen specificity and biological activity) may be used (Morrison et al. 1984 Proc Natl Acad Sci 81:6851-6855; Neuberger et al. 1984 Nature 312:604-608; Takeda et al. 1985 Nature 314:452-454).

Alternatively, techniques described for the production of single chain antibodies (U.S. Pat. No. 4,946,779) can be adapted to produce the single chain antibodies capable of binding peptides of the invention. Antibody fragments which contain specific binding sites for peptides of the invention may also be generated. Such fragments include F(ab′)2 fragments which can be produced by pepsin digestion of an antibody molecule and Fab fragments which can be generated by reducing disulfide bridges of F(ab′)2 fragments. Alternatively, Fab expression libraries may be constructed to allow rapid and easy identification of monoclonal Fab fragments with the desired specificity (Huse et al. 1989 Science 256:1275-1281).

The term “chimeric antigen receptor” (CAR) means a protein that is a chimera of a T cell receptor protein and the antigen-binding sequences of an antibody. When a CAR is expressed at the surface of a T cell, the extracellular antigen-binding sequences of the CAR target the T cell to a specific antigen, and the intracellular T cell receptor segment of the CAR mediates activation of the T cell. In this way, a CAR targets an immune response against a specific antigen. This capability has therapeutic application, particularly in cancer therapy wherein the patient's own T cells are engineered to express CARs (CAR T cells) and are then used to direct an immune response against cancerous cells expressing specific antigens (Feins et al. 2019 Am J Hematol 94 (S1) S3-S9).

CARs can be produced using standard recombinant DNA techniques for generating fusion proteins. In brief, coding sequence for the antigen-binding variable regions of an antibody which binds the antigen of interest (such as a peptide of the present invention), combined into a single chain variable fragment (scFv), is placed adjacent and in-frame with a coding sequence for the T cell receptor. This combined coding sequence is incorporated into an expression vector, which is then used to express the CAR fusion protein in cells.

As is well understood in the art, the binding of antibodies and CARs to specific targets (antigens) is mediated primarily by complementarity determining regions (CDRs) in variable regions of the antibody or CAR; the specific sequence of the CDRs and variable regions determines the affinity of the antibody or CAR for different antigens. In some embodiments, the antibody or CAR is capable of specifically binding to a peptide selected from the group consisting of SEQ ID NOs 1 to 4. In some embodiments, the antibody or CAR is capable of specifically binding to SEQ ID NO 1. In some embodiments, the antibody or CAR is capable of specifically binding to SEQ ID NO 2. In some embodiments, the antibody or CAR is capable of specifically binding to SEQ ID NO 3. In some embodiments, the antibody or CAR is capable of specifically binding to SEQ ID NO 4.

The term “capable of binding” means that the antibody or CAR binds to a peptide of the invention with higher affinity than it does to other antigens. Techniques for measuring binding affinity of antibodies and CARs for antigens are well known in the art (Jarmoskaite et al. 2020 eLife 9 e57264 DOI: 10.7554/eLife.57264), and include surface plasmon resonance (SPR) and isothermal calorimetry (ITC). The binding affinity of the antibody or CAR for a peptide of the invention, as determined by such techniques, may be expressed as the equilibrium dissociation constant (K_{D}), which is the ratio between the association rate constant (kₒₙ) and the dissociation rate constant (k_{off}) between the antibody/CAR and the antigen (e.g. peptide of the invention). The lower the K_{D} value, the higher the affinity between the antibody/CAR and the antigen. In some embodiments, the antibody or CAR is capable of binding to one or more peptides selected from the group consisting of SEQ ID NOs 1 to 4 with a KD of 1 μM or less, 100 nM or less, 10 nM or less, 1 nM or less, 100 pM or less, or 10 pM or less.

CAR T cell

The invention provides a CAR T cell expressing a CAR of the invention. The term “CAR T cell” refers to a T cell with has been engineered to express a CAR. CAR T cells can be generated from a T cell population by standard recombinant DNA and genetic manipulation techniques known in the art (see for example Roddie et al. 2019 Cytoptherapy 21 (3) 327-340). Typically, T cells are obtained from a sample from a subject suffering from the condition to be treated. The T cells are engineered to express a CAR targeting an antigen related to the condition (e.g. an antigen that is over-expressed in tumour cells in the subject). The CAR T cells thus generated are then administered to the subject. CAR T cells are thus a form of personalised medicine, as the subject's own (modified) cells are used as the therapeutic.

In some embodiments, the CAR T cell is a CAR regulatory T cell (referred to herein as a “CAR Treg cell”). In other words, in such embodiments, a regulatory T cell has been engineered to express the CAR of the invention. Regulatory T cells (Tregs) are a subset of T cells with the ability to suppress an immune response by inhibiting proliferation of and cytokine secretion by other T cells. Tregs can be isolated from a sample from a subject using techniques known in the art. The suppressive effect of Tregs can be directed towards specific targets by expression in the Treg of a CAR that recognises a particular antigen. Thus, in the present invention, expression in a Treg of a CAR that binds to the amino acid sequences of any of SEQ ID NOs 1, 2, 3 and 4 directs the CAR Treg towards those sequences, thereby suppressing an immune response against proteins comprising those sequences. The CAR Treg of the invention is thus useful in suppressing an autoimmune response to self-antigens comprising the sequences of the peptides of the invention.

### Pharmaceutical or Immunomodulatory Composition

The invention further provides a pharmaceutical or immunomodulatory composition comprising:
one or more peptides of the invention; and/or
one or more polynucleotides of the invention; and/or
one more vectors of the invention; and/or
one or more antibodies or CARs of the invention; and/or
one or more CAR T cells of the invention;
   and a pharmaceutically acceptable carrier, vehicle, diluent or excipient.

The pharmaceutical or immunomodulatory composition may comprise one type of component of the invention of those listed above, or a combination of such components. In some embodiments, the composition comprises one or more peptides of the invention and a pharmaceutically acceptable carrier, vehicle, diluent or excipient. In some embodiments, the composition comprises one or more polynucleotides of the invention and a pharmaceutically acceptable carrier, vehicle, diluent or excipient. In some embodiments, the composition comprises one more vectors of the invention and a pharmaceutically acceptable carrier, vehicle, diluent or excipient. In some embodiments, the composition comprises one or more antibodies of the invention and a pharmaceutically acceptable carrier, vehicle, diluent or excipient. In some embodiments, the composition comprises one or more CARs of the invention and a pharmaceutically acceptable carrier, vehicle, diluent or excipient. In some embodiments, the composition comprises one or more CAR T cells of the invention and a pharmaceutically acceptable carrier, vehicle, diluent or excipient. In some embodiments, the composition comprises any pairwise combination of one or more peptides of the invention, one or more polynucleotides of the invention, one more vectors of the invention, one or more antibodies or CARs of the invention, and one or more CAR T cells of the invention; as well as a pharmaceutically acceptable carrier, vehicle, diluent or excipient. In some embodiments, the composition comprises one or more peptides of the invention and one or more polynucleotides of the invention.

In preferred embodiments, the composition comprises one or more peptides of the invention and a pharmaceutically acceptable carrier, vehicle, diluent or excipient. In other words, the invention provides a pharmaceutical or immunogenic composition comprising a peptide selected from the group consisting of SEQ ID NOs 1, 2, 3 and 4 and variants thereof having at least 80% identity to any one of SEQ ID NOs 1, 2, 3 or 4; and a pharmaceutically acceptable carrier, vehicle, diluent or excipient. In some embodiments, the invention provides a pharmaceutical or immunogenic composition comprising a peptide selected from SEQ ID NO 1 and variants having at least 80% thereto; and a pharmaceutically acceptable carrier, vehicle, diluent or excipient. In some embodiments, the invention provides a pharmaceutical or immunogenic composition comprising a peptide selected from SEQ ID NO 2 and variants having at least 80% thereto; and a pharmaceutically acceptable carrier, vehicle, diluent or excipient. In some embodiments, the invention provides a pharmaceutical or immunogenic composition comprising a peptide selected from SEQ ID NO 3 and variants having at least 80% thereto; and a pharmaceutically acceptable carrier, vehicle, diluent or excipient. In some embodiments, the invention provides a pharmaceutical or immunogenic composition comprising a peptide selected from SEQ ID NO 4 and variants having at least 80% thereto; and a pharmaceutically acceptable carrier, vehicle, diluent or excipient.

The term “immunomodulatory” means that the composition is capable of inducing or suppressing an immune response when administered to a subject.

In some embodiments, the composition is an immunogenic composition. The term “immunogenic” means that the composition is capable of inducing an immune response when administered to a subject. The immune response may be a response directly against the peptides of the invention comprised in the composition, or to said peptides as expressed from the polynucleotides or vectors of the invention comprised in the composition. In other words, the immune system of the subject may recognise the peptide(s) and mount an immune response thereto. Thus, in some embodiments, the immunogenic composition comprises one or more peptides of the invention; and/or one or more polynucleotides of the invention; and/or one more vectors of the invention; and a pharmaceutically acceptable carrier, vehicle, diluent or excipient. Alternatively, the immune response may be mediated by the antibodies, CARs or CAR T cells comprised in the composition. For example, a CAR T cell of the invention comprised in the composition may recognise an antigen present in the subject (such as the sequence of a peptide of the invention within the COVID-19 virus spike protein or envelope protein in a subject suffering from COVID-19) and initiate an immune response. Thus, in some embodiments, the immunogenic composition comprises one or more antibodies or CARs of the invention; and/or one or more CAR T cells of the invention.

In some embodiments, the composition is an immunosuppressive composition. The term “immunosuppressive” means that the composition is capable of suppressing an immune response when administered to a subject. Suppression of the immune response may be mediated by the antibodies, CARs or CAR T cells comprised in the composition. For example, a CAR Treg of the invention comprised in the composition may recognise an antigen present in the subject, such as the sequence of a peptide of the invention within a self-antigen, and suppress an immune response to that self-antigen. Thus, in some embodiments, the immunosuppressive composition comprises one or more antibodies or CARs of the invention; and/or one or more CAR Tregs of the invention.

A person skilled in the art can readily formulate the pharmaceutical or immunomodulatory composition as required, depending on the components of the invention included in the composition and the intended purpose of the composition. The pharmaceutical or immunomodulatory composition comprises a pharmaceutically acceptable carrier, vehicle, diluent or excipient. The term “pharmaceutically acceptable” means that the carrier, vehicle, diluent or excipient is suitable for the intended use, route of administration and dosage of the composition and complies with standard pharmaceutical requirements (such as being non-toxic). The person skilled in the art can readily determine the appropriate carrier, vehicle, diluent or excipient to use according to the intended use of the composition.

Examples of suitable excipients for the various different forms of pharmaceutical or immunogenic compositions of the invention may be found in the Handbook of Pharmaceutical Excipients, 2nd Edition, (1994), Edited by A Wade and PJ Weller. Acceptable carriers or diluents are well known in the pharmaceutical art, and are described, for example, in Remington's Pharmaceutical Sciences, Mack Publishing Co. (A. R. Gennaro edit. 1985). Examples of suitable carriers include lactose, starch, glucose, methyl cellulose, magnesium stearate, mannitol, sorbitol. Examples of suitable diluents include ethanol, glycerol and water.

The pharmaceutical or immunogenic compositions of the invention may comprise as, or in addition to, the carrier, vehicle, diluent or excipient any suitable binder(s), lubricant(s), suspending agent(s), coating agent(s) and/or solubilising agent(s). Examples of suitable binders include starch, gelatin, natural sugars such as glucose, anhydrous lactose, freeflow lactose, beta-lactose, corn sweeteners, natural and synthetic gums, such as acacia, tragacanth or sodium alginate, carboxymethyl cellulose and polyethylene glycol. Examples of suitable lubricants include sodium oleate, sodium stearate, magnesium stearate, sodium benzoate, sodium acetate, sodium chloride. Preservatives, stabilizers, dyes and even flavouring agents may be provided in the pharmaceutical composition. Examples of preservatives include sodium benzoate, sorbic acid and esters of p-hydroxybenzoic acid. Antioxidants and suspending agents may be also included in the composition. The immunogenic composition of the invention may also comprise an adjuvant as described herein, such as AS03.

In some embodiments, the pharmaceutical or immunogenic composition is to be administered orally, enterally, rectally, parenterally, vaginally, intrabronchially, nasally, buccally or sublingually. The composition may be an edible composition, meaning the composition is approved for human or animal consumption. In some embodiments, the pharmaceutical or immunogenic compositions is be injected, such as intravenously, intraarterially, intraarticularly, intrauterinely, intrathecally, subcutaneously, intradermally, intraperitoneally, subcutaneously, perorally, muscosally or intramuscularly. The person skilled in the art can readily formulate the composition of the invention to be suitable for any route of administration. Accordingly, the pharmaceutical or immunogenic composition of the invention may be in any suitable form. Forms of the composition suitable for oral administration include compressed tablets, pills, tablets, gels, drops, pastes and capsules. The composition may alternatively be in the form of a solution or emulsion suitable for injection. The pharmaceutical or immunogenic compositions, may also be in form of suppositories, pessaries, suspensions, emulsions, pastes, lotions, ointments, creams, gels, sprays, solutions or dusting powders. The composition may be formulated to be suitable for administration via skin patch. The composition may be formulated in unit dosage form, i.e. in the form of discrete portions containing a unit dose, or a multiple or sub-unit of a unit dose.

A person of ordinary skill in the art can easily determine without undue experimentation an appropriate dose of the peptide, polynucleotide, vector, antibody, CAR and/or CAR T cell of the invention (the active) to include in a pharmaceutical or immunogenic composition of the invention that will be administered to a subject. Typically, a medical practitioner will determine the actual dosage which will be most suitable for an individual patient, and it will depend on a variety of factors including activity of the active, stability and length of action of the active, age, body weight, general health, sex and diet of the subject, mode and time of administration, rate of excretion, any drug combination, severity of the particular condition, and the individual undergoing therapy.

In some embodiments, the pharmaceutical or immunomodulatory composition of the invention comprises tolerogenic dendritic cells loaded with one or more peptide of the invention. Dendritic cells can be generated ex vivo from peripheral blood of subjects then administered to the subject (see for example Phillips et al. 2019 Frontiers in Immunology Vol 10 Article 148). Loading with peptides of the invention targets the tolerogenic dendritic cells to suppress an autoimmune response towards those antigen comprising sequences matching those peptides.

In some embodiments, the pharmaceutical or immunomodulatory composition of the invention is a tolerogenic vaccine. Tolerogenic vaccines treat disease by suppressing an autoimmune response in a subject. Examples of tolerogenic vaccines are known in the art for treatment of diseases such as multiple sclerosis (see for example Moorman et al. 2021 Frontiers in Immunology Vol 12 Article 657768). In some embodiments, the tolerogenic vaccine comprises one or more peptide of the invention. In some embodiments, the tolerogenic vaccine comprises one or CAR T cells of the invention, in particular one or more CAR Tregs of the invention.

### Kit

The invention also provides a kit comprising:
a composition of the invention; and/or
a vector of the invention; and/or
an MHC-multimer, antibody or CAR of the invention; and/or
a pharmaceutical or immunogenic composition of the invention;
   and optionally instructions.

Kits of the invention may be used to carry out any of the methods of the invention. Accordingly, in some embodiments, the kit comprises instructions for administration to a subject, for purposes of carrying out the methods of inducing an immune response or methods of treatment of the invention. In some embodiments, the kit comprises instructions for determining the presence or absence in a sample of peptides capable of binding to the antibodies or CARs. In some embodiments, the kit comprises instructions for carrying out the method of the invention for determining in a sample a level of antigen-specific T cells. In some embodiments, the kit comprises instructions for carrying out the diagnostic method or the predictive method of the invention. The kit may comprise any other components necessary to carry out any of the methods of the invention.

### Modulating an Immune Response

The invention also provides a composition of the invention, a vector of the invention, an antibody or CAR of the invention, a CAR T cell of the invention or a pharmaceutical or immunomodulatory composition of the invention for use in modulating an immune response in a subject.

The invention also provides a method of modulating an immune response in a human or animal, said method comprising administering to the subject a composition of the invention, a vector of the invention, an antibody or CAR of the invention, a CAR Treg cell of the invention or a pharmaceutical composition of the invention.

The term “modulating an immune response” refers to increasing or decreasing the strength of an immune response to an antigen, such as increasing or decreasing T cell proliferation in response to the antigen or increasing or decreasing cytokine secretion by T cells in response to the antigen. In some embodiments, “modulating an immune response” means suppressing an immune response. In some embodiments, “modulating an immune response” means enhancing an immune response.

In preferred embodiments, the invention provides a CAR Treg of the invention for use in suppressing an immune response in a subject. In preferred embodiments, the invention provides a method of suppressing an immune response in a human or animal, said method comprising administering to the subject a CAR Treg of the invention.

Treatment with Antibodies, CARs and CAR T Cells

The invention also provides an antibody or CAR of the invention, a CAR T cell of the invention, or a pharmaceutical or immunogenic composition of the invention comprising one or more antibodies or CARs of the invention and/or one or more CAR T cells of the invention, for use in the treatment or prevention of an inflammatory condition in a subject.

The invention also provides a method of treating or preventing an inflammatory condition in a subject, said method comprising administering to the subject an antibody or CAR of the invention, a CAR T cell of the invention, or a pharmaceutical or immunogenic composition of the invention comprising one or more antibodies or CARs of the invention and/or one or more CAR T cells of the invention.

The invention also provides an antibody or CAR of the invention, a CAR T cell of the invention, or a pharmaceutical or immunogenic composition of the invention comprising one or more antibodies or CARs of the invention and/or one or more CAR T cells of the invention, for use in treating COVID-19 in a subject.

The invention also provides a method of treating COVID-19 in a subject, said method comprising administering to the subject an antibody or CAR of the invention, a CAR T cell of the invention, or a pharmaceutical or immunogenic composition of the invention comprising one or more antibodies or CARs of the invention and/or one or more CAR T cells of the invention.

As described herein, the peptides of the invention are derived from COVID-19 virus envelope protein and spike protein. The antibodies, CARs and CAR T cells of the invention, which are capable of binding the peptides of the invention, may therefore be capable of initiating, mediating, and/or propagating an immune response against an infection with COVID-19 virus. Accordingly, the antibodies, CARs and CAR T cells of the invention, and pharmaceutical compositions comprising same, may be used to treat COVID-19.

COVID-19, the COVID-19 virus and means for diagnosing a subject as suffering from COVID-19 are defined and described elsewhere herein.

The person skilled in the art can readily prepare antibodies, CARs, CAR T cells and compositions to be used in treating a subject using techniques known in the art. For example, the person skilled in the art can readily determine what other components (such as carriers, vehicles, diluents, excipients etc.) should be included in a composition for administration to a subject, and what dose of antibody, CAR and/or CAR T cell should be administered, as described herein.

### MHC-Multimer

The invention provides a major histocompatibility complex (MHC)-multimer comprising a peptide of the invention. In other words, the invention provides an MHC-multimer comprising a peptide selected from the group consisting of SEQ ID NOs 1, 2, 3 and 4, and variants thereof having at least 80% identity to any one of SEQ ID NOs 1, 2, 3 or 4.

An MHC molecule is a cell surface protein encoded by polymorphic genes at the MHC locus in the vertebrate genome and expressed in all nucleated cells and platelets. MHC molecules are a critical part of the immune system as they display at the cell surface peptide fragments of proteins (antigen-presentation), where they are recognised by the T cell receptor (TCR) of T cells. In this way, T cells are exposed to foreign antigens, which can elicit an immune response, and also recognise self-antigens, avoiding inappropriate autoimmune responses. An MHC molecule may be of MHC class I (MHCI) or MHC class II (MHCII). MHCI and MHCII present different types of peptides on different cell types as part of antigen-presentation.

MHC molecules have been developed into research and diagnostic tools, referred to in the art and herein as “MHC-multimers”, which allow staining and detection of antigen-specific T cells (Dolton et al. 2015 Immunology 146 11-22). Briefly, the interaction between a peptide-MHC complex and a TCR is naturally weak and transient, but avidity of the peptide-MHC complex for TCRs can be increased by multimerising the peptide-MHC complex using biotin-streptavidin labelling to form a multimer (typically a tetramer) of peptide-MHC complexes. Thus, the term “MHC-multimer” as used herein refers to multiple peptide-MHC complexes linked to form a multimer suitable for use in staining of antigen-specific T cells. In some embodiments, the MHC-multimer is an MHC-tetramer (i.e. four peptide-MHC complexes linked to form a tetramer). In some embodiments, the MHC-multimer is an MHC-pentamer (i.e. five peptide-MHC complexes linked to form a pentamer).

MHC-multimers can theoretically be tailored to identify T cells specific for any peptide antigen (i.e. T cells expressing a TCR specific for that peptide antigen) simply by including the peptide in the MHC-multimer. MHC-multimers which can be tailored by addition of a peptide of interest (such as the peptides of the invention) are commercially available (for example from Immunaware and Biolegend). In some embodiments, the MHC-multimer comprises SEQ ID NO 1 or a variant thereof having at least 80% identity to SEQ ID NO 1. In some embodiments, the MHC-multimer comprises SEQ ID NO 2 or a variant thereof having at least 80% identity to SEQ ID NO 2. In some embodiments, the MHC-multimer comprises SEQ ID NO 3 or a variant thereof having at least 80% identity to SEQ ID NO 3. In some embodiments, the MHC-multimer comprises SEQ ID NO 4 or a variant thereof having at least 80% identity to SEQ ID NO 4.

Such MHC-multimers can be used to stain cell samples in a manner analogous to an antibody, as the MHC-multimer binds to TCRs specific for the peptide incorporated in the MHC-multimer. MHC-multimers conjugated to fluorophores can be detected using flow cytometry, alongside antibody staining of other cell surface proteins, thereby allowing detailed characterisation of T cell populations. MHC-multimers may alternatively be linked to rare metal ions, allowing their detection by mass spectrometry (Newell et al. 2013 Nat Biotechnol 31 623-629). Thus, in some embodiments, the MHC-multimer is conjugated to a label. In some embodiments, the label is a fluorophore. In some embodiments, the MHC-multimer is conjugated to a fluorophore. In some embodiments, the label is a rare metal ion. MHC-multimers may be constructed from MHCI or MHCII molecules. Thus, in some embodiments, the MHC-multimer comprises MHCI molecules. In some embodiments, the MHC-multimer comprises MHCII molecules.

### Method for Determining Level of Antigen-Specific T Cells

The invention provides a method for determining in a sample a level of antigen-specific T cells expressing a T cell receptor (TCR) that binds to a peptide of the invention, the method comprising the steps of
(a) incubating the sample with one or more peptide of the invention; and
(b) determining the level of antigen-specific T cells in the sample by detecting binding of the one or more peptide to the TCR of antigen-specific T cells in the sample.

In other words, the invention provides a method for determining in a sample a level of antigen-specific T cells expressing a T cell receptor (TCR) that binds to a peptide selected from the group consisting of SEQ ID NO 1, 2, 3 and 4, and variants thereof having at least 80% identity to any one of SEQ ID NOs 1, 2, 3 or 4, the method comprising the steps of
(a) incubating the sample with one or more peptide selected from the group consisting of SEQ ID NO 1, 2, 3 and 4, and variants thereof having at least 80% identity to any one of SEQ ID NOs 1, 2, 3 or 4; and
(b) determining the level of antigen-specific T cells in the sample by detecting binding of the one or more peptide to the TCR of antigen-specific T cells in the sample.

The term “antigen-specific T cell” refers to a T cell that expresses a TCR that binds to a peptide of the invention (i.e. SEQ ID NO 1, 2, 3 or 4, or variant having at least 80% identity to any one of SEQ ID NOs 1, 2, 3 or 4). In some embodiments, the antigen-specific T cell expresses a TCR that binds to SEQ ID NO 1 or a variant thereof having at least 80% identity to SEQ ID NO 1. In some embodiments, the antigen-specific T cell expresses a TCR that binds to SEQ ID NO 2 or a variant thereof having at least 80% identity to SEQ ID NO 2. In some embodiments, the antigen-specific T cell expresses a TCR that binds to SEQ ID NO 3 or a variant thereof having at least 80% identity to SEQ ID NO 3. In some embodiments, the antigen-specific T cell expresses a TCR that binds to SEQ ID NO 4 or a variant thereof having at least 80% identity to SEQ ID NO 4. In some embodiments, the antigen-specific T cell expresses a TCR that binds to SEQ ID NO 1. In some embodiments, the antigen-specific T cell expresses a TCR that binds to SEQ ID NO 2. In some embodiments, the antigen-specific T cell expresses a TCR that binds to SEQ ID NO 3. In some embodiments, the antigen-specific T cell expresses a TCR that binds to SEQ ID NO 4. In some embodiments, the antigen-specific T cell expresses a TCR that binds to a peptide selected from the group consisting of SEQ ID NOs 2, 3 and 4 and variants thereof having at least 80% identity to any one of SEQ ID NOS 2, 3 or 4.

“Incubating the sample with one or more peptides of the invention” means maintaining the one or more peptides in the presence of the sample for a period and under conditions sufficient for the one or more peptides to bind to the TCR. The person skilled in the art can readily determine an appropriate period and conditions. The sample may be incubated with any one of the peptides of the invention, or with multiple peptides of the invention, either simultaneously or sequentially. As will be appreciated, the precise peptides that are incubated with the sample determines which antigen-specific T cells will be detected in step (b) of the method. More particularly, the antigen-specific T cells detected in step (b) are those which express a TCR that binds to the peptide(s) with which the sample was incubated in step (a) because it is the binding of the peptides to the TCR of the antigen-specific T cells which marks those cells.

In some embodiments, the sample is incubated with SEQ ID NO 1 or a variant thereof having at least 80% identity to SEQ ID NO 1. In some embodiments, the sample is incubated with SEQ ID NO 2 or a variant thereof having at least 80% identity to SEQ ID NO 2. In some embodiments, the sample is incubated with SEQ ID NO 3 or a variant thereof having at least 80% identity to SEQ ID NO 3. In some embodiments, the sample is incubated with SEQ ID NO 4 or a variant thereof having at least 80% identity to SEQ ID NO 4. In some embodiments, the sample is incubated with SEQ ID NO 1. In some embodiments, the sample is incubated with SEQ ID NO 2. In some embodiments, the sample is incubated with SEQ ID NO 3. In some embodiments, the sample is incubated with SEQ ID NO 4. In some embodiments, the sample is incubated with a peptide selected from the group consisting of SEQ ID NOs 2, 3 and 4 and variants thereof having at least 80% identity to any one of SEQ ID NOs 2, 3 or 4.

In some embodiments, the sample is incubated with more than one peptide of the invention, such as two, three or four peptides of the invention. In other words, the sample is incubated with one of the peptides of the invention alongside one or more other peptides of the invention. For example, in some embodiments, the sample is incubated with a peptide having the sequence of SEQ ID NO 2, a peptide having the sequence of SEQ ID NO 3 and a peptide having the sequence of SEQ ID NO 4.

The method for determining in a sample a level of antigen-specific T cells of the invention comprises a step of determining a level of antigen-specific T cells in a sample. “Determining a level of antigen-specific T cells” means measuring, either quantitatively or semi-quantitatively, the amount of antigen-specific T cells in the sample. Accordingly, “determining a level of antigen-specific T cells” is not limited to quantifying the level of antigen-specific T cells, and thus encompasses simply determining presence or absence of the antigen-specific T cells in the sample. Typically, the determination will reveal the absolute level of antigen-specific T cells in the sample, or the level of antigen-specific T cells relative to the level of a reference value.

The level of antigen-specific T cells may be determined more than once in a given sample, for example for the purpose of statistical calculations. Alternatively, or in addition, a level may be determined one or more times in more than one sample obtained from the subject.

In some embodiments, “determining a level of antigen-specific T cells” means determining an absolute or approximate number of antigen-specific T cells.

In some embodiments, “determining a level of antigen-specific T cells” means determining a proportion of antigen-specific T cells. In some embodiments, the proportion of antigen-specific T cells is the proportion of T cells in the sample that are antigen-specific (i.e. express a TCR that binds to a peptide of the invention). In other words, the method determines what proportion of T cells in the sample, out of all T cells in the sample, are antigen-specific T cells. In some embodiments, the proportion of antigen-specific T cells is the proportion of all mononuclear cells in the sample that are antigen-specific T cells. In other words, the method determines what proportion of mononuclear cells in the sample are antigen-specific T cells. In some embodiments, the proportion of antigen-specific T cells is the proportion of all cells in the sample that are antigen-specific T cells. In other words, the method determines what proportion, out of all cells in the sample, are antigen-specific T cells. In any of these embodiments, the proportion of antigen-specific T cells may be expressed as a percentage.

In some embodiments, the one or more peptide is comprised in one or more MHC-multimer of the invention. Thus, in some embodiments, the invention provides a method for determining in a sample a level of antigen-specific T cells TCR that binds to a peptide of the invention, the method comprising the steps of
(a) incubating the sample with one or more MHC-multimer of the invention; and
(b) determining the level of antigen-specific T cells in the sample by detecting binding of the MHC-multimer to the TCR of antigen-specific T cells in the sample.

Put another way, the invention provides a method for staining antigen-specific T cells with the MHC-multimers of the invention.

In some embodiments, the sample is incubated with an MHC-multimer comprising SEQ ID NO 1 or a variant thereof having at least 80% identity to SEQ ID NO 1. In some embodiments, the sample is incubated with an MHC-multimer comprising SEQ ID NO 2 or a variant thereof having at least 80% identity to SEQ ID NO 2. In some embodiments, the sample is incubated with an MHC-multimer comprising SEQ ID NO 3 or a variant thereof having at least 80% identity to SEQ ID NO 3. In some embodiments, the sample is incubated with an MHC-multimer comprising SEQ ID NO 4 or a variant thereof having at least 80% identity to SEQ ID NO 4.

In some embodiments, the sample is incubated with more than one MHC-multimer of the invention, such as two, three or four MHC-multimers of the invention. In other words, the sample is incubated with an MHC-multimer comprising one of the peptides of the invention alongside one or more other MHC-multimers, each comprising a different peptide of the invention. For example, in some embodiments, the sample is incubated with an MHC-multimer comprising SEQ ID NO 2 or a variant thereof having at least 80% identity to SEQ ID NO 2, an MHC-multimer comprising SEQ ID NO 3 or a variant thereof having at least 80% identity to SEQ ID NO 3 and an MHC-multimer comprising SEQ ID NO 4 or a variant thereof having at least 80% identity to SEQ ID NO 4.

### Detection of Peptide Binding TCR

The level of antigen-specific T cells in the sample is determined by detecting binding of the one or more peptide to the TCR of antigen-specific T cells in the sample. The detection may be carried out by any appropriate means known in the art. Detection of binding may be direct or indirect. Direct detection of binding means that the actual binding of the peptide to the TCR is detected. Examples of direct detection of binding of the peptide are immunoprecipitation and flow cytometry. As part of the detection process, the peptide may be cross-linked to the TCR. Indirect detection of binding means that the binding of the peptide to the TCR is detected by measuring an effect caused by binding of the peptide to the TCR. Examples of indirect detection of binding of the peptide to the TCR are ELIspot assay and T cell proliferation assay.

In some embodiments, binding of the one or more peptide to the TCR of antigen-specific T cells in the sample is detected by flow cytometry. Flow cytometry is widely used in the art and essentially entails characterising features of cells in a sample by detecting fluorescence (see for example Cossarizza et al. 2019 Eur J Immunol 49 (1) 1457-1973 and Mckinnon Curr Protoc Immunol 2019 120:5.1.1-5.1.11). In a typical example of a flow cytometry protocol, a sample of cells is stained with a primary antibody against a specific cell surface protein, and then with a secondary antibody conjugated to a fluorescent moiety. The sample is then passed through a flow cytometer, wherein the cells flow individually past excitatory lasers and a fluorescence detector. In this manner, the number of fluorescent cells (and so the number of cells expressing the protein of interest) can be counted, and sorted if required. Antibodies and other reagents for use in flow cytometry to detect cMet-positive T cells are known in the art and commercially available (see for example Komarowska et al. Immunity 2015 42, 1087-1099). Accordingly, in some embodiments of the methods of the invention, the level of cMet-positive T cells is determined by flow cytometry. Flow cytometry may also be referred to as fluorescence-activated cell sorting (FACS). Determination of the level of cMet-positive T cells by flow cytometry has the particular advantages of being faster than existing methods of diagnosis, with the time required to stain a whole blood sample from a subject for flow cytometry being less than 1 hour, such as about 52 minutes or as low as 30 minutes. In some embodiments, the level of cMet-positive T cells is determined using flow cytometry by staining the cells with labelled antibodies to cMet. In some embodiments, the level of cMet-positive T cells is determined using flow cytometry by staining the cells with labelled cMet ligand, preferably labelled HGF.

Use of flow cytometry to detect binding of the peptide of the invention to the TCR is particularly preferred when the peptide is comprised in an MHC-multimer of the invention. Thus, in preferred embodiments, the invention provides a method for determining in a sample a level of antigen-specific T cells TCR that binds to a peptide of the invention, the method comprising the steps of
(a) incubating the sample with one or more MHC-multimer of the invention; and
(b) determining the level of antigen-specific T cells in the sample by detecting binding of the MHC-multimer to the TCR of antigen-specific T cells in the sample using flow cytometry.

In such embodiments, the MHC-multimer is typically labelled with a fluorophore.

In some embodiments, binding of the one or more peptide to the TCR of antigen-specific T cells in the sample is detected by mass spectrometry. Mass spectrometry may be used particularly to detect MHC-multimers of the invention labelled with a rare metal ion.

In some embodiments, binding of the one or more peptide to the TCR of antigen-specific T cells in the sample is detected by ELISpot (enzyme-linked immunospot) assay. ELISpot assay is widely used in the art to quantify antigen-specific T cells in a sample by measuring secretion of cytokines upon T cell activation (Slota et al. 2011 Expert Rev Vaccines 10 (3) 299-306). In brief, cytokine-specific antibodies are immobilised on a support (e.g. multi-well plate) and the sample is incubated with the immobilised antibodies in the presence of the activating agent (such as a peptide of the present invention). This incubation corresponds to step (a) of the method of the invention for determining a level of antigen-specific T cells (incubating the sample with one or more peptide of the invention). Antigen-specific T cells in the sample activated by the peptide secrete cytokines which are captured by the immobilised antibodies. After removal of the cells and washing, further cytokine-specific antibodies are added and linked via biotin-streptavidin to an enzyme. A colorimetric substrate for the enzyme is added, and the intensity of colour produced is detected to enable quantification of the level of antigen-specific T cells in the sample. Reagents, such as anti-cytokine antibodies, enzymes and substrates, and kits for carrying out ELISPot assays are commercially available, for example from R&D Systems, Creative Biolabs, Immunospot and Mabtech.

In some embodiments, binding of the one or more peptide to the TCR of antigen-specific T cells in the sample is detected by T cell proliferation assay. The term “T cell proliferation assay” refers to assays used in the art to detect antigen-specific T cells by detecting proliferation of said cells. In brief, a sample is incubated with the activating agent, such as a peptide of the present invention. This incubation corresponds to step (a) of the method of the invention for determining a level of antigen-specific T cells (incubating the sample with one or more peptide of the invention). An agent that indicates cell proliferation is then added to the sample and, after a period to allow proliferation of T cells in response to the peptide, the agent is measured. Examples of such agents include 3H-thymidine, a radio-labelled nucleotide which is incorporated into newly synthesised DNA and can be detected by measuring radioactivity of the sample, and carboxyfluorescein succinimidyl ester (CSFE), a fluorescent agent that is diluted as cells divide and can be measured using flow cytometry.

### Diagnostic Method—Antigen-Specific T Cells

The invention provides a method for diagnosing a condition in a subject, the method comprising the steps of:
(a) determining a level of antigen-specific T cells in a sample obtained from the subject using the method for determining in a sample a level of antigen-specific T cells of the invention; and
(b) comparing the level of antigen-specific T cells in the sample to an antigen-specific T cell reference value,
   wherein the level of antigen-specific T cells in the sample compared to the antigen-specific T cell reference value is indicative of the presence or absence of the condition in the subject.

In other words, the invention provides a method for diagnosing a condition in a subject, the method comprising the steps of:
(a) determining a level of antigen-specific T cells expressing a TCR that binds to a peptide selected from the group consisting of SEQ ID NO 1, 2, 3 and 4 and variants thereof having at least 80% identity to any one of SEQ ID NOs 1, 2, 3 or 4 in a sample obtained from the subject, by
   (i) incubating the sample with one or more peptides selected from the group consisting of SEQ ID NO 1, 2, 3 and 4 and variants thereof having at least 80% identity to any one of SEQ ID NOS
   (ii) determining the level of antigen-specific T cells in the sample by detecting binding of the one or more peptide to the TCR of antigen-specific T cells in the sample; and
(b) comparing the level of antigen-specific T cells in the sample to an antigen-specific T cell reference value,
   wherein the level of antigen-specific T cells in the sample compared to the antigen-specific T cell reference value is indicative of the presence or absence of the condition in the subject.

These methods, and specific embodiments of these methods, are referred to herein as “diagnostic methods of the invention” or “methods of diagnosis of the invention”.

Similarly, the invention provides a method for predicting whether a subject will develop a condition, the method comprising the steps of:
(a) determining a level of antigen-specific T cells in a sample obtained from the subject using the method for determining in a sample a level of antigen-specific T cells of the invention; and
(b) comparing the level of antigen-specific T cells in the sample to an antigen-specific T cell reference value, wherein the level of antigen-specific T cells in the sample compared to the antigen-specific T cell reference value is predictive of whether the subject will develop the condition.

In other words, the invention provides a method for predicting whether a subject will develop a condition, the method comprising the steps of:
(a) determining a level of antigen-specific T cells in a sample obtained from the subject expressing a TCR that binds to a peptide selected from the group consisting of SEQ ID NO 1, 2, 3 and 4 and variants thereof having at least 80% identity to any one of SEQ ID NOs 1, 2, 3 or 4 by
   (i) incubating the sample with one or more peptides selected from the group consisting of SEQ ID NO 1, 2, 3 and 4 and variants thereof having at least 80% identity to any one of SEQ ID NOs 1, 2, 3 or 4; and
   (ii) determining the level of antigen-specific T cells in the sample by detecting binding of the one or more peptide to the TCR of antigen-specific T cells in the sample; and
(b) comparing the level of antigen-specific T cells in the sample to an antigen-specific T cell reference value,
   wherein the level of antigen-specific T cells in the sample compared to the antigen-specific T cell reference value is predictive of whether the subject will develop the condition.

These methods, and specific embodiments of these methods, are referred to herein as “predictive methods of the invention” or “methods of prediction of the invention”.

The method of diagnosis and method of prediction of the invention each comprise a step of determining a level of antigen-specific T cells in a sample obtained from the subject using the method of determining a level of antigen-specific T cells of the invention, which is described in detail elsewhere herein.

Diagnostic Method—cMet-Positive T Cells

In some embodiments of the diagnostic methods of the invention and the predictive methods of the invention, the method further comprises the steps of:
(c) determining a level of cMet-positive T cells in a sample obtained from the subject; and
(d) comparing the level of cMet-positive T cells in the sample to a cMet-positive T cell reference value,
   wherein the level of cMet-positive T cells in the sample compared to the cMet-positive T cell reference value is indicative of the presence or absence of the condition in the subject or predictive of whether the subject will develop the condition.

Thus, in preferred embodiments, the invention provides a method for diagnosing a cardiac inflammatory condition in a subject, the method comprising the steps of:
(a) determining a level of antigen-specific T cells in a sample obtained from the subject using the method for determining in a sample a level of antigen-specific T cells of the invention; and
(b) comparing the level of antigen-specific T cells in the sample to an antigen-specific T cell reference value,
(c) determining a level of cMet-positive T cells in a sample obtained from the subject; and
(d) comparing the level of cMet-positive T cells in the sample to a cMet-positive T cell reference value,
   wherein the level of antigen-specific T cells in the sample compared to the antigen-specific T cell reference value, and/or the level of cMet-positive T cells in the sample compared to the cMet-positive T cell reference value, is indicative of the presence or absence of the condition in the subject.

In some embodiments, presence or absence of the condition in the subject is indicated by either the level of antigen-specific T cells in the sample compared to the antigen-specific T cell reference value or the level of cMet-positive T cells in the sample compared to the cMet-positive T cell reference value (i.e. if either comparison indicates the presence of the condition, then the condition is considered to be present in the subject). In other embodiments, presence or absence of the condition in the subject is indicated by the level of antigen-specific T cells in the sample compared to the antigen-specific T cell reference value and the level of cMet-positive T cells in the sample compared to the cMet-positive T cell reference value (i.e. the condition is considered to be present in the subject only if both comparisons indicate the presence of the condition).

“Determining a level of cMet-positive T cells” means measuring, either quantitatively or semi-quantitatively, the amount of cMet-positive T cells in a sample from the subject. Typically, the determination will reveal the absolute level of cMet-positive T cells in a sample from a subject, or the level of a cMet-positive T cells relative to the level of the cMet-positive T cell reference value.

In some embodiments, “determining a level of cMet-positive T cells” means determining an absolute or approximate number of cMet-positive T cells.

In some embodiments, “determining a level of cMet-positive T cells” means determining a proportion of cMet-positive T cells. In some embodiments, the proportion of cMet-positive T cells is the proportion of T cells in the sample that express cMet. In other words, the method determines what proportion of T cells in the sample, out of all T cells in the sample, are cMet-positive T cells. In some embodiments, the proportion of cMet-positive T cells is the proportion of all mononuclear cells in the sample that express cMet. In other words, the method determines what proportion of all mononuclear cells in the sample are cMet-positive T cells. In some embodiments, the proportion of cMet-positive T cells is the proportion of all cells in the sample that express cMet. In other words, the method determines what proportion of all cells in the sample are cMet-positive T cells. In any of these embodiments, the proportion of cMet-positive T cells may be expressed as a percentage.

cMet-positive T cells may be detected using flow cytometry, as described herein.

In some embodiments of the invention, the step of determining a level of cMet-positive T cells is carried out:
(a) before the onset of a cardiac inflammatory condition in the subject; and/or
(b) whilst the subject is showing symptoms of a cardiac inflammatory condition; and/or
(c) during and/or after the use of a treatment, therapy or prophylaxis for the cardiac inflammatory condition; and/or
(d) whilst the subject is showing symptoms of COVID-19; and/or
(e) during and/or after the subject has been treated with an anti-COVID-19 agent.

### Reference Value

In the methods of diagnosis and prediction of the present invention, the level of antigen-specific T cells in the sample is compared to an antigen-specific T cell reference value. In some embodiments of the methods of diagnosis and prediction of the present invention, the level of cMet-positive T cells in the sample is compared to a cMet-positive T cell reference value.

A “reference value” (i.e. either an antigen-specific T cell reference value or a cMet-positive T cell reference value) may include, but is not limited to, a value obtained from a reference subject (and samples obtained therefrom), or pre-determined absolute values.

Typically, a reference value is derived from a healthy subject, a subject known to be suffering from a particular condition or disease, or a subject who has recovered from a particular condition or disease. In the context of the present invention, the reference value may be derived from one or more of:
(a) a subject who has never suffered from a cardiac inflammatory condition;
(b) a subject who has recovered from a cardiac inflammatory condition;
(c) a subject who has never been infected with COVID-19 virus;
(d) a subject who has recovered from COVID-19;
(e) a subject who has not been vaccinated against COVID-19; and
(f) a subject who has been vaccinated against COVID-19 and did not develop a cardiac inflammatory condition.

The reference value may be, for example, a predetermined measurement of a level of antigen-specific T cells or cMet-positive T cells which is present in a sample from a normal subject, i.e. a subject who is not suffering from a cardiac inflammatory condition or any of (a) to (f) above. The reference value may, for example, be based on a mean or median level of antigen-specific T cells or cMet-positive T cells in a control population of subjects, e.g. 5, 10, 100, 1000 or more subjects (who may either be age- and/or gender-matched or unmatched to the test subject) who show no symptoms of a cardiac inflammatory condition.

The reference value may be determined using corresponding methods to the determination of level of antigen-specific T cells or cMet-positive T cells in the test sample, e.g. using one or more samples taken from a control population of subjects. For instance, in some embodiments levels of antigen-specific T cells or cMet-positive T cells in reference value samples may be determined in parallel assays to the test samples. In alternative embodiments, the reference value may have been previously determined, or may be calculated or extrapolated, without having to perform a corresponding determination on a reference value with respect to each test sample obtained.

In one embodiment, the reference value is derived from a subject suffering from a cardiac inflammatory condition. In one embodiment, the reference value is derived from the same subject, but at an earlier time. Thus, the invention may enable the status of a subject, such as disease progression in a subject, to be monitored over time. In particular, this embodiment finds utility when monitoring the response to therapy for the cardiac inflammatory condition over time.

In some embodiments, the reference value is a range of values. For example, it may be determined that healthy subjects present levels of antigen-specific T cells or cMet-positive T cells within a particular “healthy” range. Equally, subjects suffering from a cardiac inflammatory condition may present levels of antigen-specific T cells or cMet-positive T cells within a particular “disease” range. Reference values, and in particular ranges of values, may be optimised over time as more data are obtained and analysed.

### Comparing to Reference Value

The methods of diagnosis and prediction of the invention comprise comparing the level of antigen-specific T cells in the sample to an antigen-specific T cell reference value. Furthermore, in some embodiments, the methods of diagnosis and prediction of the invention comprise comparing the level of cMet-positive T cells in the sample to a cMet-positive T cell reference value.

In embodiments comprising determining an absolute or approximate number of antigen-specific T cells or cMet-positive T cells, the reference value is also an absolute number of antigen-specific T cells or cMet-positive T cells respectively. In other words, in such embodiments, “comparing the level of antigen-specific T cells in the sample to an antigen-specific T cell reference value” means comparing the absolute or approximate number of antigen-specific T cells determined to be in the sample to an absolute number of antigen-specific T cells that is the antigen-specific T cell reference value, and “comparing the level of cMet-positive T cells in the sample to a cMet-positive T cell reference value” means comparing the absolute or approximate number of cMet-positive T cells determined to be in the sample to an absolute number of cMet-positive T cells that is the cMet-positive T cell reference value.

In embodiments comprising determining a proportion of antigen-specific T cells or cMet-positive T cells, the reference value is also a proportion of antigen-specific T cells or cMet-positive T cells respectively. In other words, in such embodiments, “comparing the level of antigen-specific T cells in the sample to an antigen-specific T cell reference value” means comparing the proportion of antigen-specific T cells determined to be in the sample to a proportion of antigen-specific T cells that is the antigen-specific T cell reference value, and “comparing the level of cMet-positive T cells in the sample to a cMet-positive T cell reference value” means comparing the proportion of cMet-positive T cells determined to be in the sample to a proportion of cMet-positive T cells that is the cMet-positive T cell reference value. In some embodiments, the antigen-specific T cell reference value is a proportion of T cells that are antigen-specific. In some embodiments, the cMet-positive T cell reference value is a proportion of T cells that are cMet-positive. In some embodiments, the antigen-specific T cell reference value is a proportion of mononuclear cells that are antigen-specific. In some embodiments, the cMet-positive T cell reference value is a proportion of mononuclear cells that are cMet-positive. In some embodiments, the antigen-specific T cell reference value is a proportion of all cells that are antigen-specific. In some embodiments, the cMet-positive T cell reference value is a proportion of all cells that are cMet-positive.

In the diagnostic methods of the invention, the level of antigen-specific T cells in the sample compared to the antigen-specific T cell reference value is indicative of the presence or absence of the condition in the subject.

Similarly, in some embodiments of the diagnostic methods of the invention, the level of cMet-positive T cells in the sample compared to the reference value is indicative of the presence or absence of the condition in the subject.

“Indicative of the presence or absence of the condition” means the act of diagnosis of a condition in a subject (i.e. positive diagnosis), or the act of ruling out a diagnosis of a condition in a subject (i.e. negative diagnosis).

The level of antigen-specific T cells and/or cMet-positive T cells in the sample may be indicative of the presence or absence of the condition. For example, the level of antigen-specific T cells and/or cMet-positive T cells may be merely suggestive, or may definitively denote the presence or absence of the condition in a subject. Thus, in some embodiments, the level of antigen-specific T cells is suggestive of the presence or absence of the condition. Thus, in some embodiments, the level of cMet-positive T cells is suggestive of the presence or absence of the condition. In some embodiments, the level of antigen-specific T cells denotes the presence or absence of the cardiac inflammatory condition. In some embodiments, the level of cMet-positive T cells denotes the presence or absence of the cardiac inflammatory condition.

In the methods of prediction of the invention, the level of antigen-specific T cells in the sample compared to the antigen-specific T cell reference value is predictive of whether the subject will develop the condition.

Similarly, in some embodiments of the methods of prediction of the invention, the level of cMet-positive T cells in the sample compared to the cMet-positive reference value is predictive of whether the subject will develop the condition.

“Predictive of whether the subject will develop the condition” means the act of predicting that the subject will develop the condition (i.e. positive prediction), or the act of predicting that the subject will not develop the condition (i.e. negative prediction).

The level of antigen-specific T cells and/or cMet-positive T cells in the sample may be predictive of the presence or absence of the condition. For example, the level of antigen-specific T cells and/or cMet-positive T cells may be merely suggestive of a positive prediction, or may definitively denote that the subject will develop the condition. Thus, in some embodiments, the level of antigen-specific T cells is suggestive that the subject will develop the condition. In some embodiments, the level of cMet-positive T cells is suggestive that the subject will develop the condition. In some embodiments, the level of antigen-specific T cells denotes that the subject will develop the condition. In some embodiments, the level of cMet-positive T cells denotes that the subject will develop the condition.

In some embodiments of the diagnostic method of the invention, an increased level of antigen-specific T cells in the sample compared to the antigen-specific T cell reference value is indicative of the presence of the condition in the subject.

Likewise, in some embodiments of the method of prediction of the invention, an increased level of antigen-specific T cells in the sample compared to the antigen-specific T cell reference value is indicative of the presence of the condition in the subject or predictive that the subject will develop the condition.

Thus, in some embodiments, a higher absolute number of antigen-specific T cells in the sample compared to the number of antigen-specific T cells that is the reference value is indicative or predictive of the presence of the condition in the subject. In some embodiments, a higher approximate number of antigen-specific T cells in the sample compared to the number of antigen-specific T cells that is the antigen-specific T cell reference value is indicative or predictive of the presence of the condition in the subject. In some embodiments, a higher proportion of antigen-specific T cells in the sample compared to the antigen-specific T cell reference value is indicative or predictive of the presence of the condition in the subject.

In some embodiments of the diagnostic method of the invention, an increased level of cMet-positive T cells in the sample compared to the cMet-positive T cell reference value is indicative of the presence of the condition in the subject.

Likewise, in some embodiments of the method of prediction of the invention, an increased level of cMet-positive T cells in the sample compared to the cMet-positive T cell reference value is predictive that the subject will develop the condition.

Thus, in some embodiments, a higher absolute number of cMet-positive T cells in the sample compared to the number of cMet-positive T cells that is the reference value is indicative or predictive of the presence of the condition in the subject. In some embodiments, a higher approximate number of cMet-positive T cells in the sample compared to the number of cMet-positive T cells that is the reference value is indicative or predictive of the presence of the condition in the subject. In some embodiments, a higher proportion of cMet-positive T cells in the sample compared to the reference value is indicative or predictive of the presence of the condition in the subject.

For example, a level of antigen-specific T cells in the sample of more than 1% of the total T cells in the sample may be indicative of the presence of the condition. In this case, the antigen-specific T cell reference value is 1% of the total T cells in the sample. Similarly, a level of cMet-positive T cells in the sample of more than 1% of the total T cells in the sample may be indicative of the presence of the condition. In this case, the cMet-positive T cell reference value is 1% of the total T cells in sample.

In some embodiments, the level of antigen-specific T cells in the sample differs by at least 1%, at least 5%, at least 10%, at least 15%, at least 20%, at least 30%, at least 50%, at least 75% or at least 100% compared to the antigen-specific T cell reference value. In some embodiments, the level of antigen-specific T cells in the sample differs by at least 2-fold, for example at least 3-fold, 4-fold, 5-fold, 6-fold, 8-fold or 10-fold compared to the antigen-specific T cell reference value. Preferably, the level of antigen-specific T cells in the sample differs by at least 2-fold compared to the antigen-specific T cell reference value.

In some embodiments, the level of cMet-positive T cells in the sample differs by at least 1%, at least 5%, at least 10%, at least 15%, at least 20%, at least 30%, at least 50%, at least 75% or at least 100% compared to the cMet-positive T cell reference value. In some embodiments, the level of cMet-positive T cells in the sample differs by at least 2-fold, for example at least 3-fold, 4-fold, 5-fold, 6-fold, 8-fold or 10-fold compared to the cMet-positive T cell reference value. Preferably, the level of cMet-positive T cells in the sample differs by at least 2-fold compared to the cMet-positive T cell reference value.

In some embodiments, an unchanged level of antigen-specific T cells is indicative of absence of the condition in the subject. By “unchanged level of antigen-specific T cells” it is meant that the relative or absolute level of the antigen-specific T cells is of substantially the same value compared to the antigen-specific T cell reference value.

In some embodiments, an unchanged level of cMet-positive T cells is indicative of absence of the condition in the subject. By “unchanged level of cMet positive T cells” it is meant that the relative or absolute level of the cMet positive T cells is of substantially the same value compared to the cMet-positive T cell reference value.

In some embodiments, an unchanged level means a level which differs by less than 2-fold, such as less than 1.5-fold, compared to the reference value.

In some embodiments, the antigen-specific T cell reference value is 0 or substantially 0. In other words, in some embodiments the antigen-specific T cell reference value is that there are no, or substantially no, antigen-specific T cells. Thus, in some embodiments, the mere presence of antigen-specific T cells in the sample is indicative of the presence of the condition in the subject. Likewise, in some embodiments, the presence of antigen-specific T cells in the sample predicts that the subject will develop the condition.

In some embodiments, the cMet-positive T cell reference value is 0 or substantially 0. In other words, in some embodiments the cMet-positive T cell reference value is that there are no, or substantially no, cMet-positive T cells. Thus, in some embodiments, the mere presence of cMet-positive T cells in the sample is indicative of the presence in the subject of the condition. Likewise, in some embodiments, the presence of cMet-positive T cells in the sample predicts that the subject will develop the condition.

As will be apparent to the person skilled in the art, the actual determination of whether a level of antigen-specific T cells or cMet-positive T cells is substantially increased, decreased or unchanged compared to a reference value may depend on the outcome of one or more statistical analyses, all of which are known and are routine to the person skilled in the art.

### Condition

The term “condition” refers to a disease or pathology from which the subject may be suffering, and which it is desirable to diagnose or predict to assist treatment, therapy, prophylaxis or management of the condition.

In some embodiments the condition is an inflammatory condition. Inflammation is part of the immune response to damage or disease, so may be caused by a variety of factors. An “inflammatory condition” may also be referred to simply as “inflammation”. Inflammatory conditions include inflammation caused by bacterial infection, viral infection or cancer. Without wishing to be bound by theory, antigen-specific T cells expressing a TCR that binds to a peptide of the invention may develop in a subject in response to a pathogenic antigen (e.g. from a viral infection) as part of the immune response to the antigen. The antigen-specific T cells themselves then mediate an inflammatory response, leading to the inflammatory condition. Thus, the antigen-specific T cells may be causative agents of the inflammatory condition, and therefore their presence (i.e. level) in a sample (such as a blood sample) from the subject may be used to diagnose or predict whether the subject is suffering from the inflammatory condition.

In some embodiments, the condition is COVID-19. “COVID-19” is defined and described in detail elsewhere herein. Without wishing to be bound by theory, antigen-specific T cells expressing a TCR that binds to a peptide of the invention may develop in a subject suffering from COVID-19 as part of the immune response to COVID-19, as antigen-specific T cells may develop against epitopes in COVID-19 virus spike protein and/or envelope protein which correspond to the peptides of the invention. Thus, the presence (i.e. level) of such antigen-specific T cells in a sample (such as a blood sample) from the subject may be used to diagnose or predict whether the subject is suffering from COVID-19.

### Cardiac Inflammatory Condition

In some embodiments, the condition is a cardiac inflammatory condition. A cardiac inflammatory condition is inflammation of the heart. The present inventors have found that administration of the peptides of the invention, which are derived from COVID-19 virus spike protein and envelope protein, to animal subjects induces inflammation of the heart. Without wishing to be bound by theory, it is hypothesised that COVID-19 virus spike protein and envelope protein in an infected subject or subject who has been vaccinated against COVID-19 induce an immune response which inadvertently also targets cardiac self-antigens, leading to cardiac inflammation. Such an immune response may be mediated by antigen-specific T cells expressing TCRs which bind to the peptides of the invention. Accordingly, the level of such antigen-specific T cells in a subject may be used as a biomarker for diagnosis of a cardiac inflammatory condition. Furthermore, the level of such antigen-specific T cells in a subject may also be used to predict that the subject will develop a cardiac inflammatory condition before other signs of cardiac inflammation become evident.

Thus, in preferred embodiments, the invention provides a method for diagnosing a cardiac inflammatory condition in a subject, the method comprising the steps of:
(a) determining a level of antigen-specific T cells in a sample obtained from the subject using the method for determining in a sample a level of antigen-specific T cells of the invention; and
(b) comparing the level of antigen-specific T cells in the sample to an antigen-specific T cell reference value,
   wherein the level of antigen-specific T cells in the sample compared to the antigen-specific T cell reference value is indicative of the presence or absence of the cardiac inflammatory condition in the subject.

Furthermore, in addition to the present inventors have identified that cardiotropic, cMet-positive T cells are increased in the blood of animal subjects immunised with the peptides of the invention. Cardiotropic, cMet-positive T cells mediate cardiac inflammation. Accordingly, the level of cMet-positive T cells in a subject may be used as a biomarker for diagnosis of a cardiac inflammatory condition. Furthermore, the level of such cMet-positive T cells in a subject may also be used to predict that the subject will develop a cardiac inflammatory condition before other signs of cardiac inflammation become evident.

Thus, in preferred embodiments, the invention provides a method for diagnosing a cardiac inflammatory condition in a subject, the method comprising the steps of:
(a) determining a level of antigen-specific T cells in a sample obtained from the subject using the method for determining in a sample a level of antigen-specific T cells of the invention; and
(b) comparing the level of antigen-specific T cells in the sample to an antigen-specific T cell reference value,
(c) determining a level of cMet-positive T cells in a sample obtained from the subject; and
(d) comparing the level of cMet-positive T cells in the sample to a cMet-positive T cell reference value,
   wherein the level of antigen-specific T cells in the sample compared to the antigen-specific T cell reference value, and/or the level of cMet-positive T cells in the sample compared to the cMet-positive T cell reference value, is indicative of the presence or absence of the cardiac inflammatory condition in the subject.

In some embodiments, the cardiac inflammatory condition is myocarditis. Myocarditis is inflammation of the myocardium (heart muscle), which can cause arrhythmia of the heart and reduce the ability of the heart to pump blood. Symptoms of myocarditis include chest pain, abdominal pain, rapid or irregular heartbeat, fainting, fatigue, shortness of breath and general weakness, fever and swelling of legs or feet.

In some embodiments, the cardiac inflammatory condition is pericarditis. Pericarditis is inflammation of the pericardium, which is a fluid-filled sac around the heart. Symptoms of pericarditis include chest pain, fever shortness of breath and a fast heartbeat.

In some embodiments, the cardiac inflammatory condition encompasses both myocarditis and pericarditis. In other words, the method of the invention may diagnose the subject as having, or predict that the subject will develop, both myocarditis and pericarditis.

In some embodiments, the cardiac inflammatory condition is endocarditis. Endocarditis is inflammation of the inner lining of the chambers and valves of the heart. Symptoms of endocarditis include chest pain, abdominal pain, shortness of breath, cough, fever, blood in urine, muscle, joint and back pain, night sweats and skin changes. In some embodiments, the cardiac inflammatory condition encompasses myocarditis, pericarditis and endocarditis.

cMet-Positive T Cells

cMet-positive T cells are effector T cells expressing the hepatocyte growth factor (HGF) receptor cMet. cMet is also known as MET, hepatocyte growth factor receptor (HGFR), tyrosine-protein kinase Met, AUTS9, RCCP2 and DFNB97. cMet is a single-membrane-pass receptor tyrosine kinase. Binding of the chemokine HGF to cMet triggers multiple downstream signalling pathways that may influence gene expression and thereby cellular phenotype and/or behaviour in a variety of ways depending on cell type. Binding of HGF to cMet on T cells during T cell activation induces cardiotropic behaviour (i.e. triggers migration of the T cells to the heart). cMet-positive T cells may also be denoted as cMet+ T cells.

In some embodiments, the cMet-positive T cells are cardiotropic T cells. “Cardiotropic” means that the T cells are attracted towards and function within the heart. In some embodiments, the cMet-positive T cells are virus-induced cardiotropic T cells. The term “virus-induced” means that the T cells have developed the cardiotropic phenotype in response to a virus, such as the COVID-19 virus.

### Other T Cell Markers-CCR4, CXCR3, CD4, CD8

In some embodiments, the cMet-positive T cells are CCR4-positive T cells. In other words, the T cells express both cMet and CCR4. Accordingly, in some embodiments the method of the invention comprises determining a level of cMet-positive, CCR4-positive T cells (i.e. cMet+CCR4+ T cells). CCR4 (CC chemokine receptor 4) is a receptor for two CC chemokine ligands (CCL17 and 22) that is expressed on T cells. CCR4 is also known as CKR4, CC-CKR4, K5-5, CD194, CMKBR4 and ChemR13.

In some embodiments, the cMet-positive T cells are CXCR3-positive T cells. In other words, the T cells express both cMet and CXCR3. Accordingly, in some embodiments the method of the invention comprises determining a level of cMet-positive, CXCR3-positive T cells (i.e. cMet+CXCR3+ T cells). CXCR3 (CXC receptor 3) is a receptor for three CXC chemokine ligands (CXCL9, 10 and 11) that is expressed on T cells. CXCR3 is also known as GPR9, MigR, CD182, CD183, CKR-L2, CMKAR3 and IP10-R.

In some embodiments, the cMet-positive T cells are cMet-positive, CCR4-positive, CXCR3-positive T cells (i.e. cMet+CCR4+CXCR3+ T cells). In other words, the T cells express all three of cMet, CCR4 and CXCR3. Accordingly, in some embodiments the method of the invention comprises determining a level of cMet-positive, CCR4-positive T cells, CXCR3-positive T cells (i.e. cMet+CCR4+CXCR3+ T cells). cMet+CCR4+CXCR3+ is a specific molecular signature expressed by cardiotropic T cells.

In some embodiments, the cMet-positive T cells are CD4-positive T cells. In other words, the T cells express both cMet and CD4. Accordingly, in some embodiments the method of the invention comprises determining a level of cMet-positive, CD4-positive T cells. CD4 (cluster of differentiation 4) is a co-receptor for the T cell receptor that is expressed on the surface of immune cells. CD4 is also known as IMD79, OKT4D and CD4mut.

In some embodiments, the cMet-positive T cells are CD8-positive T cells. In other words, the T cells express both cMet and CD8. Accordingly, in some embodiments the method of the invention comprises determining a level of cMet-positive, CD8-positive T cells. CD8 (cluster of differentiation 8) is a co-receptor for the T cell receptor that is expressed on the surface of immune cells. There are two isoforms of CD8, alpha and beta, denoted CD8a and CD8b respectively. “CD8” as used herein refers to both isoforms. In other words, CD8-positive T cells are T cells expressing either CD8a alone, CD8b alone or both CD8a and CD8b. CD8a is also known as p32 and Leu2. CD8b is nalos known as LY3, P37, LEU2, LYT3 and CD8B1.

All references herein to determining the level of cMet-positive T cells encompass determining the level of T cells that express cMet as well as any of CCR4, CXCR3, CD4, CD8, or any combination of these cell surface proteins. Thus, in some embodiments, determining a level of cMet-positive T cells means determining a level of cMet-positive, CCR4-positive T cells (i.e. detecting cells that express cMet and CCR4). In some embodiments, determining a level of cMet-positive T cells means determining a level of cMet-positive, CXCR3-positive T cells (i.e. detecting cells that express cMet and CXCR3). In some embodiments, determining a level of cMet-positive T cells means determining a level of cMet-positive, CCR4-positive, CXCR3-positive T cells (i.e. detecting cells that express cMet, CCR4 and CXCR3).

### Sequences

Amino acid and nucleotide sequences of human cMet, CCR4, CXCR3, CD4 and CD8 are available from publicly accessible databases, e.g. under the accession numbers as shown in the table below:

| | HGNC | Ensembl | UniProt | RefSeq (mRNA) | RefSeq (protein) |
|---|---|---|---|---|---|
| cMet | 7029 | ENSG00000105976 | P08581 | NM_000245.4 | NP_000236.2 |
| | | | | NM_001127500.3 | NP_001120972.1 |
| | | | | NM_001324401.3 | NP_001311330.1 |
| | | | | NM_001324402.2 | NP_001311331.1 |
| CCR4 | 1605 | ENSG00000183813 | P51679 | NM_005508.5 | NP_005499.1 |
| CXCR3 | 4540 | ENSG00000186810 | P49682 | NM_001142797.2 | NP_001136269.1 |
| | | | | NM_0010504.2 | NP_001495.1 |
| CD4 | 1678 | ENSG00000010610 | P01730 | NM_000616.5 | NP_000607.1 |
| | | | | NM_001195014.3 | NP001181943.1 |
| | | | | NM_001195015.3 | NP_001181944.1 |
| | | | | NM_001195016.3 | NP001181945.1 |
| | | | | NM_001195017.3 | NP001181946.1 |
| | | | | NM_001382705.1 | NP001369634.1 |
| | | | | NM_001382706.1 | NP001369635.1 |
| | | | | NM_001382707.1 | NP001369636.1 |
| | | | | NM_001382714.1 | NP_001369643.1 |
| CD8a | 1706 | ENSG00000153563 | P01732 | NM_001145873.1 | NP_001139345.1 |
| | | | | NM_001382698.1 | NP_001369627.1 |
| | | | | NM_001768.7 | NP_001759.3 |
| | | | | NM_171827.4 | NP_741969.1 |
| CD8b | 1707 | ENSG00000172116 | P10966 | NM_001178100.2 | NP001171571.1 |
| | | | | NM_004931.5 | NP_004922.1 |
| | | | | NM_172101.5 | NP_742099.1 |
| | | | | NM_172102.5 | NP_742100.1 |
| | | | | NM_172213.5 | NP_757362.1 |

Amino acid and nucleotide sequences corresponding to further variants and homologs of the above genes, as well as genes found in other species, may be found in similar publicly accessible databases or by identifying sequences showing homology to the above human sequences.

The present inventors have found that peptides derived from COVID-19 virus spike protein and envelope protein induce cardiac inflammation and an increase in the level of cMet-positive T cells. The sequences of the peptides, and corresponding sequence identifiers, are shown in the following table:

| Peptide | Source | | SEQ |
|---|---|---|---|
| name | protein | Sequence | ID NO |
| PA01 | envelope | MYSFVSEETGTLIVNSV | 1 |
| P87 | spike | LLHAPATVCGPKKST | 2 |
| PS556 | spike | NKKELPFQQFGRDIA | 3 |
| P177 | spike | PHGVVFLHVTYVPAQ | 4 |

The sequence of COVID-19 virus envelope protein is SEQ ID NO 5 herein. The sequence of COVID-19 virus spike protein is SEQ ID NO 6 herein.

### COVID-19

In some embodiments of the diagnostic method or the predictive method of the invention, the subject is infected with COVID-19 virus and/or suffering from one or more morbidity associated with COVID-19.

The term “COVID-19” refers to the disease caused by infection with SARS-CoV-2. The terms “COVID-19 virus” and “SARS-CoV-2” are used interchangeably herein. The genome of SARS-CoV-2 has been sequenced (Wu et al. Nature 2020 579, 265-269), and changes in the genome have been reported (Tang et al. Nat Sci Rev 7 (6): 1012-1023). SARS-CoV-2 is a coronavirus with a single-stranded positive sense RNA genome of about 30,000 nucleotides in length. The virus genome encodes four structural proteins: the spike surface glycoprotein(S), small envelope protein (E), matrix protein (M) and nucleocapsid protein (N). The spike protein binds receptors on the host cell surface and mediates membrane fusion (Wrapp et al. Science 2020 367 (6483): 1260-1263). The envelope, matrix and nucleocapsid proteins function in encasing the RNA genome and mediating virus assembly, budding and envelope formation. Full details of the molecular biology, life cycle, mechanism of infection and associated pathology of SARS-CoV-2 are readily available in the art. The term “COVID-19 virus” encompasses SARS-CoV-2 and any natural or engineered variants thereof, including particularly natural variants that may develop in the future through mutation of SARS-CoV-2.

In some embodiments, the subject is infected with COVID-19 virus. Whether a person is infected with COVID-19 virus may be readily determined using techniques known in the art (reviewed in, for example, Song et al. Lab Chip 2021 21 (9): 1634-1660), such as polymerase chain reaction (PCR) based tests to identify the presence of SARS-CoV-2 genome in the subject and immunoassays to detect antibodies against SAS-COV-2. A subject infected with COVID-19 virus may exhibit viremia (presence of COVID-19 virus in the bloodstream).

In some embodiments, the subject is suffering from one or more morbidity associated with COVID-19. A morbidity associated with COVID-19 is a symptom or condition caused directly or indirectly by infection with the COVID-19 virus. Morbidities associated with COVID-19 are known in the art and may be readily recognised by a skilled medical practitioner. Examples of symptoms of COVID-19, which are considered morbidities associated with COVID-19 in the context of the present invention, include high temperature, fever or chills, cough, sore throat, shortness of breath or difficulty breathing, chest pain, fatigue, headache, loss or change to sense of smell or taste, nausea or vomiting and diarrhoea. Other morbidities associated with COVID-19 include pneumonia, acute respiratory distress, lung sepsis and cardiac inflammation, such as myocarditis or pericarditis.

In some embodiments, the subject is both infected with the COVID-19 virus and suffering from one or more morbidity associated with COVID-19. In other embodiments, the subject is not infected with the COVID-19 virus but is suffering from one or more morbidity associated with COVID-19. In such embodiments, the subject may have previously been infected with COVID-19 virus and since cleared the infection but is still suffering from one or more morbidity associated with COVID-19. Thus, in some embodiments, the subject is suffering from one or more morbidity associated with a prior COVID-19 infection in the subject. In other embodiments, the subject is infected with the COVID-19 virus but is not noticeably suffering from any morbidity associated with COVID-19. In other words, the subject may be asymptomatic.

In some embodiments of the invention, the subject is suffering from long COVID. The symptoms and conditions found in long COVID cases may be considered morbidities associated with COVID-19. In other words, in some embodiments, a subject suffering from one or more morbidity associated with COVID-19 is suffering from long COVID. Long COVID may also be referred to as “post COVID-19 condition”. Long COVID is a set of persistent conditions experienced by some subjects that have been infected with SAS-COV-2. The World Health Organisation definition states that long COVID (post COVID-19 condition) occurs in individuals with a history of probable or confirmed SARS-CoV-2 infection, usually 3 months from the onset of COVID-19 with symptoms that last for at least 2 months and cannot be explained by an alternative diagnosis. Common symptoms include fatigue, shortness of breath, cognitive dysfunction but also others and generally have an impact on everyday functioning. Symptoms may be new onset following initial recovery from an acute COVID-19 episode or persist from the initial illness. Symptoms may also fluctuate or relapse over time (WHO-A clinical case definition of post COVID-19 condition by a Delphi consensus, 6 Oct. 2021).

### Vaccination Against COVID-19

In some embodiments of the diagnostic method or predictive method of the invention, the subject has been vaccinated against COVID-19. In some embodiments, the subject is both infected with COVID-19 virus and has been vaccinated against COVID-19. In some embodiments, the subject is both suffering from one or more morbidity associated with COVID-19 and has been vaccinated against COVID-19. In some embodiments, the subject is infected with COVID-19 virus, suffering from one or more morbidity associated with COVID-19 and has been vaccinated against COVID-19.

Cardiac inflammation has been reported after vaccination with mRNA-based COVID-19 vaccines, in particular the Moderna vaccine (mRNA-1273) and Pfizer/BioNTech vaccine (BNT162b2). Without wishing to be bound by theory, it is hypothesised that COVID-19 virus spike protein and envelope protein in a subject who has been vaccinated against COVID-19 induce an immune response which inadvertently also targets cardiac self-antigens, leading to cardiac inflammation. Such an immune response may be mediated by antigen-specific T cells expressing TCRs which bind to the peptides of the invention. Accordingly, the level of such antigen-specific T cells in a subject may be used as a biomarker for diagnosis of a condition in a subject who has been vaccinated against COVID-19. Furthermore, the level of such antigen-specific T cells in a subject may also be used to predict that a subject who has been vaccinated against COVID-19 will develop a condition.

Additionally, cardiac inflammation is mediated by cardiotropic, cMet-positive T cells. Accordingly, the level of cMet-positive T cells in a subject who has been recently vaccinated against COVID-19 may be used as a biomarker for diagnosis of a condition. Furthermore, the level of such cMet-positive T cells in a subject who has been recently vaccinated against COVID-19 may also be used to predict that the subject will develop a condition.

In some embodiments, the subject has been vaccinated against COVID-19 within 6 weeks prior to performing said method of diagnosis. In some embodiments, the subject has been vaccinated against COVID-19 within 6, 5, 4, 3, 2 or 1 weeks prior to carrying out the method of diagnosis. In some embodiments, the subject has been vaccinated against COVID-19 within 56 days prior to carrying out the method of diagnosis. In some embodiments, the subject has been vaccinated against COVID-19 within 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 days prior to carrying out the method of diagnosis. Preferably, the subject has been vaccinated against COVID-19 within 30, 21, 14, 10 or 7 days prior to carrying out the method of diagnosis.

In some embodiments, the subject has been vaccinated against COVID-19 within 6 weeks prior to carrying out the method of prediction. In some embodiments, the subject has been vaccinated against COVID-19 within 6, 5, 4, 3, 2 or 1 weeks prior to carrying out the method of prediction. In some embodiments, the subject has been vaccinated against COVID-19 within 56 days prior to carrying out the method of prediction. In some embodiments, the subject has been vaccinated against COVID-19 within 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 days prior to carrying out the method of prediction. Preferably, the subject has been vaccinated against COVID-19 within 30, 21, 14, 10 or 7 days prior to carrying out the method of prediction.

The expression “vaccinated against COVID-19” means that a vaccine intended to provide at least some degree of immunity against COVID-19 has been administered to the subject. Such vaccines are referred to herein as “COVID-19 vaccines”. Subjects vaccinated with any COVID-19 vaccine, including vaccines not authorised or developed or at the date of filing of the present application, are within the scope of the invention. In some embodiments, the subject has been vaccinated against COVID-19 using a vaccine selected from the following: BNT162b2 (the vaccine), mRNA-1273 (the Moderna vaccine), AZD1222 (the Pfizer/BioNTech Oxford/AstraZeneca vaccine), the Sputnik V vaccine, AD5-nCOV (Convidecia), Ad26.COV2.S (the Janssen vaccine), CoronaVac (the Sinovac vaccine), BBIBP-CorV, WIBP-CorV, Covaxin, EpiVacCorona, ZF2001 (ZIFIVAX) and MVC-COV1901.

In some embodiments, the subject has been vaccinated against COVID-19 using an mRNA vaccine. An “mRNA vaccine” is a vaccine wherein the active ingredient is a nucleoside-modified mRNA encoding a component of the COVID-19 virus. Examples of mRNA vaccines are BNT162b2 and mRNA-1273.

In some embodiments of the invention, the subject has been vaccinated against COVID-19 using BNT162b2 vaccine. Other names for BNT162b2 include “the Pfizer/BioNtech COVID-19 vaccine”, “the Pfizer/BioNtech vaccine” or “the Pfizer vaccine” (as it was developed by the companies Pfizer and BioNTech), “Comirnaty” (which is its brand name) and “tozinameran”. BNT162b2 is an mRNA vaccine as the active ingredient is a nucleoside-modified mRNA encoding the viral spike glycoprotein of the COVID-19 virus. Full details of BNT162b2 are readily available in the art (see for example Polack et al. N Engl J Med 2020 383:2603-2615).

In some embodiments, the subject has been vaccinated against COVID-19 using mRNA-1273 vaccine. Other names for mRNA-1273 include “the Moderna COVID-19 vaccine” or simply “the Moderna vaccine” (as it was developed by the company Moderna), “Spikevax” (which is its brand name), “CX-024414” and “TAK-919”. mRNA-1273 is an mRNA vaccine as the active ingredient is a nucleoside-modified mRNA encoding the viral spike glycoprotein of the COVID-19 virus. Full details of mRNA-1273 are readily available in the art (see for example Baden et al. N Engl J Med 2021 384:403-416).

Some COVID-19 vaccines are administered as a single dose (e.g. the Janssen vaccine), whilst other COVID-19 vaccines require two administrations (i.e. two doses) separated by a number of weeks or months (e.g. the Pfizer/BioNTech vaccine) to induce immunity against COVID-19. A COVID-19 vaccine may also be administered as a “booster”, meaning that the vaccine is administered to a subject who has already been administered the requisite number of doses to acquire immunity against COVID-19. A booster dose is typically administered months after the original vaccination doses. The present invention encompasses all of these types of administration. That is, in some embodiments, the vaccination of the subject against COVID-19 within 30 days prior to carrying out the method of diagnosis or prediction is the single vaccination required to confer immunity, the first vaccination of two intended vaccinations, the second vaccination of two vaccinations or a booster vaccination. Where a subject has been vaccinated against COVID-19 multiple times, the period of “within 30 days prior to carrying out the method of diagnosis/prediction” is measured from the most recent vaccination. Myocarditis has been observed to be more common after the second of two vaccinations. Thus, in some embodiments, the vaccination is the second of two vaccinations against COVID-19.

### Subject

In some embodiments, the subject is a human. The subject may be any age, gender or ethnicity. The subject may be a human adult or human child. In the context of the present invention, a “human adult” is a human subject of 16 years of age or older at the time of sampling, and a “human child” is a human subject of less than 16 years of age at the time of sampling.

In some embodiments, the subject is a male human. In some embodiments, the subject is from 10 to 60 years of age. In some embodiments, the subject is from 16 to 40 years of age. In some embodiments, the subject is from 16 to 30 years of age. In some embodiments, the subject is from 16 to 21 years of age. In some embodiments, the subject is from 16 to 19 years of age.

The subject may exhibit one or more symptoms of COVID-19. The subject may exhibit one or more symptoms of a cardiac inflammatory condition. The subject may have been previously characterised as having a cardiac inflammatory condition by other diagnostic methods. Where the results of previous tests are ambiguous or inconclusive, the method of the present invention may be used to confirm the diagnosis.

The subject may have a predisposition to develop a cardiac inflammatory condition. For example, there may be an increased risk or likelihood that the subject will develop a cardiac inflammatory condition at some point in the future.

### Sample

The term “sample” as used herein may refer to a sample used in the method of the invention for determining a level of antigen-specific T cells, or to a sample used in the diagnostic method or predictive method of the invention. As the diagnostic method and predictive method of the invention comprise the method for determining a level of antigen-specific T cells comprised in of the invention, it will be appreciated that the sample may be the same in all three of these types of method of the invention. However, it will also be appreciated that when the method of the invention for determining a level of antigen-specific T cells is used in isolation (i.e. not as part of the diagnostic method or predictive method of the invention), the sample used may be the same or may be different from a sample that would be used in the diagnostic method or predictive method of the invention. A sample used in the method of the invention for determining a level of antigen-specific T cells may or may not be appropriate for use in the diagnostic method or predictive method of the invention.

In some embodiments, the sample is a sample of cultured cells. In some embodiments (such as the diagnostic and predictive methods of the invention), the sample is a sample obtained from a subject. In such embodiments, the sample is not returned to the subject after the method of the invention has been carried out. The sample may also be referred to as “the test sample” herein. The sample may be or may be derived from an ex vivo sample.

In preferred embodiments, the sample obtained from the subject is whole blood or a fraction of whole blood. Preferably, the sample is peripheral whole blood. Peripheral whole blood is the circulating pool of blood, as opposed to that sequestered in the lymphatic system or organs. In some embodiments, the sample is processed prior to being subjected to the method of diagnosis or prediction of the invention. For example, peripheral blood mononuclear cells (PBMCs) may be isolated from peripheral blood obtained from a subject, and the PBMCs subjected to the method of diagnosis or prediction of the invention. Accordingly, in some embodiments, the sample is PBMCs obtained from the subject. PBMCs can be isolated from peripheral blood by density gradient centrifugation, as known in the art (Boyum Scand J Clin Lab Invest Suppl 1968 97:77-89). In some embodiments, the cells are fixed prior to being subjected to a method of the invention.

Combination with Further Diagnostic Techniques

The diagnostic and predictive methods of the invention may be used in combination with existing methods for diagnosis of cardiac inflammatory conditions. Such existing methods to diagnose cardiac inflammation include electrocardiogram (detecting the pattern of electrical activity of the heart), echocardiogram (detecting sounds waves reflected from the beating heart), chest X-ray (to assess heart morphology and presence of fluid in or around the heart), cardiac MRI (to assess heart function and morphology, and myocardial tissue characteristics), cardiac catheterization and heart muscle biopsy, and blood tests to check for proteins typically elevated in cardiac inflammation due to cardiac damage, such as cardiac troponin I (cTnI), cardiac troponin T (cTnT), b-type natriuretic peptide (BNP) and N-terminal proBNP (NT-proBNP). The person skilled in the art is familiar with, and can readily carry out, these existing diagnostic methods (see for example Siripanthong et al. Heart Rhythm 2020 17 (9): 1463-1471).

Thus, in some embodiments, the methods of the invention comprise an additional step of diagnosing a cardiac inflammatory condition in the subject using one or more diagnostic methods selected from the following: electrocardiogram, echocardiogram, chest X-ray, cardiac magnetic resonance imaging (MRI), cardiac catheterization, heart muscle biopsy, determining a level of cardiac troponin I (cTnI) in a blood sample from the subject, determining a level of cardiac troponin T (cTnT) in a blood sample from the subject, determining a level of b-type natriuretic peptide (BNP) in a blood sample from the subject, and determining a level of N-terminal proBNP (NTproBNP) in a blood sample from the subject. The level of cTnI in the sample may be compared to a cTnI reference value, wherein the level of cTnI in the sample compared to the cTnI reference value is indicative of the presence or absence of the cardiac inflammatory condition in the subject. The level of cTnT in the sample may be compared to a cTnT reference value, wherein the level of cTnT in the sample compared to the cTnT reference value is indicative of the presence or absence of the cardiac inflammatory condition in the subject. The level of BNP in the sample may be compared to a BNP reference value, wherein the level of BNP in the sample compared to the BNP reference value is indicative of the presence or absence of the cardiac inflammatory condition in the subject. The level of NTproBNP in the sample may be compared to a NTproBNP reference value, wherein the level of NTproBNP in the sample compared to the NTproBNP reference value is indicative of the presence or absence of the cardiac inflammatory condition in the subject.

### Therapy of Cardiac Inflammatory Condition

The present invention also provides methods of treating a cardiac inflammatory condition in a subject. In particular, the invention provides a method of treating a subject suffering from a cardiac inflammatory condition, the method comprising the steps of:
(a) diagnosing a cardiac inflammatory condition using the method for diagnosing a cardiac inflammatory condition of the invention; and
(b) administering to the patient a therapeutically effective amount of a treatment, therapy or prophylaxis for a cardiac inflammatory condition.

Thus, the invention provides a method of treating a subject suffering from a cardiac inflammatory condition, the method comprising the steps of:
(a) diagnosing a cardiac inflammatory condition in a subject, the diagnosis comprising the steps of:
   (i) determining a level of antigen-specific T cells in a sample obtained from the subject using the method for determining in a sample a level of antigen-specific T cells of the invention; and
   (ii) comparing the level of antigen-specific T cells in the sample to a antigen-specific T cell reference value,
   wherein the level of antigen-specific T cells in the sample compared to the antigen-specific T cell reference value is indicative of the presence or absence of the cardiac inflammatory condition in the subject; and
(b) administering to the patient a therapeutically effective amount of a treatment, therapy or prophylaxis for a cardiac inflammatory condition.

The method of treating the cardiac inflammatory condition in a subject may further comprise a step (c) repeating step (a) after administration of the treatment, therapy or prophylaxis. If the cardiac inflammatory condition is still present (as compared to that determined in step (a)), the method for treating the cardiac inflammatory condition in a subject may further comprise a step (d) administering a therapeutically effective amount of an alternative treatment, therapy or prophylaxis for a cardiac inflammatory condition to the subject, wherein the alternative treatment, therapy or prophylaxis differs from the treatment, therapy or prophylaxis administered in step (b).

The invention also provides a therapeutically effective amount of a treatment, therapy or prophylaxis for a cardiac inflammatory condition for use in a method of treating a subject suffering from a cardiac inflammatory condition, wherein the patient has been diagnosed with a cardiac inflammatory condition using the method for diagnosing a cardiac inflammatory condition of the invention.

Thus, the invention also provides a therapeutically effective amount of a treatment, therapy or prophylaxis for a cardiac inflammatory condition for use in a method of treating a subject suffering from a cardiac inflammatory condition, wherein the patient has been diagnosed with a cardiac inflammatory condition using a method for diagnosing a cardiac inflammatory condition in a subject, the method comprising the steps of:
(a) determining a level of antigen-specific T cells in a sample obtained from the subject using the method for determining in a sample a level of antigen-specific T cells of the invention; and
(b) comparing the level of antigen-specific T cells in the sample to an antigen-specific reference value, wherein the level of antigen-specific T cells in the sample compared to the antigen-specific T cell reference value is indicative of the presence or absence of the cardiac inflammatory condition in the subject.

The method of treating a subject suffering from a cardiac inflammatory condition may further comprise, in diagnostic step (a), as described herein:
(iii) determining a level of cMet-positive T cells in a sample obtained from the subject; and
(iv) comparing the level of cMet-positive T cells in the sample to a cMet-positive T cell reference value,
   wherein the level of cMet-positive T cells in the sample compared to the cMet-positive T cell reference value is indicative of the presence or absence of the condition in the subject or predictive of whether the subject will develop the condition. In other words, the diagnostic method used in the method of treatment may comprise determining a level of cMet-positive T cells as described herein.

The treatment, therapy or prophylaxis for a cardiac inflammatory condition may be any suitable agent that can treat or alleviate the signs and/or symptoms of the cardiac inflammatory condition. The treatment, therapy or prophylaxis can be one or more treatment, therapy or prophylaxis that may be administered over a time course and/or simultaneously or at different times.

In some embodiments, the treatment, therapy or prophylaxis for a cardiac inflammatory condition is one or more of: a glucokinase activator, an antiviral drug, an inhibitor of effector T cells, an activator of regulatory T cells, a steroidal or non-steroidal immunosuppressive drug such as an anti-inflammatory biologic, colchicine, a beta-blocker, an angiotensin converting enzyme (ACE) inhibitor, an angiotensin receptor blocker (ARB), a mineralocorticoid antagonist, a neprolysin inhibitor, a sodium-glucose transport protein 2 (SGLT2) inhibitor, a diuretic, an antibiotic or intravenous immunoglobulin (IVIG).

An example of a glucokinase activator for use in the invention is piragliatin. Examples of antiviral drugs for use in the invention include Lagevrio (molnupiravir) and Paxlovid (PF-07321332/ritonavir). Example of inhibitors of effector T cells for use in the invention include methylprednisolone and tocilizumab (anti-IL-6). Examples of activators of regulatory T cells for use in the invention include low-dose rapamycin and Tregalizumab (a non-depleting IgG1 mAb that binds to a unique epitope of CD4 and selectively induces Treg activation). Examples of immune suppressive drugs for use in the invention include cyclosporin, tacrolimus, azathioprine and mycophenolate mofetil. Examples of steroid drugs for use in the invention include prednisolone, methylprednisolone and hydrocortisone. Examples of non-steroidal anti-inflammatory drugs for use in the invention include aspirin and ibuprofen. Examples of anti-inflammatory biologics include monoclonal antibodies directed to components of the immune system including membrane-bound targets and receptors, extracellular proteins and cytokines. Examples of beta-blockers for use in the invention include acebutolol and propranolol. Examples of ACE inhibitors for use in the invention include benazepril and captopril. Examples of ARBs for use in the invention include irbesartan, valsartan, losartan and candesartan. Examples of mineralocorticoid antagonists for use in the invention include spironolactone and eplerenone. Examples of neprolysin inhibitors for use in the invention include sacubitril combined with the ARB inhibitor valsartan in the formulation known as Entresto. Examples of SGLT2 inhibitors include canagliflozin, dapagliflozin and empagliflozin. An example of a diuretic for use in the invention is furosemide. An example of an antibiotic for use in the invention is clarithromycin.

Suitable dosage amounts and regimens of the treatment, therapy or prophylaxis for a cardiac inflammatory condition to be used in conjunction with the present invention may be adequately determined by the person skilled in the art. For example, a treatment, therapy or prophylaxis may be formulated and administered to a subject in any suitable composition for the treatment of a cardiac inflammatory condition. In particular embodiments, an effective amount of a treatment, therapy or prophylaxis is administered to the subject. In this context, the term “effective amount” or “therapeutically effective amount” means an amount effective, at dosages and for periods of time necessary to achieve the desired result, for example, to treat the cardiac inflammatory conditions.

The treatment, therapy or prophylaxis may be administered to a subject using a variety of techniques. For example, the treatment, therapy or prophylaxis may be administered systemically, which includes by injection including intramuscularly or intravenously, orally, sublingually, transdermally, subcutaneously, intranasally. Alternatively, the treatment, therapy or prophylaxis may be administered directly at a site affected by the cardiac inflammatory condition.

The concentration and amount of the treatment, therapy or prophylaxis to be administered will typically vary, depending on, for example, the severity of the cardiac inflammatory condition, the type of treatment, therapy or prophylaxis that is administered, the mode of administration, the age and health of the subject, and the like.

The treatment, therapy or prophylaxis may be an agent formulated in a pharmaceutical composition together with a pharmaceutically acceptable carrier, vehicle, excipient or diluent. The compositions may routinely contain pharmaceutically acceptable concentrations of salt, buffering agents, preservatives and various compatible carriers. For instance the agent may be formulated in a physiological buffer solution.

The proportion and identity of the pharmaceutically acceptable carrier, vehicle, excipient or diluent may be determined by the chosen route of administration, compatibility with live cells, and standard pharmaceutical practice. Generally, the pharmaceutical composition will be formulated with components that will not significantly impair the biological properties of the agent. Suitable carriers, vehicles, excipients and diluents are described, for example, in Remington's Pharmaceutical Sciences (Remington's Pharmaceutical Sciences, Mack Publishing Company, Easton, Pa., USA 1985).

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, amino acid sequences are written left to right in amino to carboxy orientation, respectively.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

The publications discussed herein are provided solely for their disclosure prior to the filing date of the present application. Nothing herein is to be construed as an admission that such publications constitute prior art to the claims appended hereto. All publications mentioned in the above specification are herein incorporated by reference.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in immunology, molecular biology, virology, cardiac pathology or related fields are intended to be within the scope of the following claims.

The invention will now be further described by way of Examples, which are meant to serve to assist one of ordinary skill in the art in carrying out the invention and are not intended in any way to limit the scope of the invention.

### EXAMPLES

### Example 1—Identification of COVID Peptides

The sequences of the SARS-CoV-2 coronavirus envelope and spike proteins were compared to human protein sequences to identify regions of sequence identity. This comparison identified four amino acid sequences (one within the envelope protein sequence and three within the spike protein sequence) that have identity to sequences in human proteins. The four amino acid sequences (“COVID peptides”) are shown in Table 1.

TABLE 1

| Peptide | Source | | SEQ |
|---|---|---|---|
| name | protein | Sequence | ID NO |
| PA01 | envelope | MYSFVSEETGTLIVNSV | 1 |
| P87 | spike | LLHAPATVCGPKKST | 2 |
| PS556 | spike | NKKELPFQQFGRDIA | 3 |
| P177 | spike | PHGVVFLHVTYVPAQ | 4 |

The full length sequences of SARS-CoV-2 coronavirus envelope protein (SEQ ID NO 5) and spike protein (SEQ ID NO 6) are shown below, with the location of the peptides shown by shading.

SARS-CoV-2 envelope protein
(SEQ ID NO 5)
MYSFVSEETG TLIVNSVLLF FAFVVELLVT LAILTALRLC AYCCNIVNVS LVKPTVYVYS
RVKNLNSSEG VPDLLV
SARS-CoV-2 spike protein
(SEQ ID NO 6)
MFVFLVLLPL VSSQCVNLTT RTQLPPAYTN SFTRGVYYPD KVFRSSVLHS TQDLFLPFFS
NVTWFHAIHV SGTNGTKRFD NPVLPFNDGV YFASTEKSNI IRGWIFGTTL DSKTQSLLIV
NNATNVVIKV CEFQFCNDPF LGVYYHKNNK SWMESEFRVY SSANNCTFEY VSQPFLMDLE
GKQGNFKNLR EFVFKNIDGY FKIYSKHTPI NLVRDLPQGF SALEPLVDLP IGINITRFQT
LLALHRSYLT PGDSSSGWTA GAAAYYVGYL QPRTELLKYN ENGTITDAVD CALDPLSETK
CTLKSFTVEK GIYQTSNFRV QPTESIVREP NITNLCPFGE VFNATRFASV YAWNRKRISN
CVADYSVLYN SASFSTEKCY GVSPTKLNDL CFTNVYADSF VIRGDEVRQI APGQTGKIAD
YNYKLPDDFT GCVIAWNSNN LDSKVGGNYN YLYRLFRKSN LKPFERDIST EIYQAGSTPC
NGVEGFNCYF PLQSYGFQPT NGVGYQPYRV VVLSFELLHA PATVCGPKKS TNLVKNKCVN
FNFNGLTGTG VLTESNKKFL PFQQFGRDIA DTTDAVRDPQ TLEILDITPC SFGGVSVITP
GTNTSNQVAV LYQNVNCTEV PVAIHADQLT PTWRVYSTGS NVFQTRAGCL IGAEHVNNSY
ECDIPIGAGI CASYQTQTNS PSRAGSVASQ SIIAYTMSLG AENSVAYSNN SIAIPTNFTI
SVTTEILPVS MTKTSVDCTM YICGDSTECS NLLLQYGSFC TQLNRALTGI AVEQDKNTQE
VFAQVKQIYK TPPIKDFGGF NFSQILPDPS KPSKRSFIED LLFNKVTLAD AGFIKQYGDC
LGDIAARDLI CAQKFNGLTV LPPLLTDEMI AQYTSALLAG TITSGWTFGA GPALQIPFAM
QMAYRFNGIG VTQNVLYENQ KLIANQFNSA IGKIQDSLSS TPSALGKLQD VVNQNAQALN
TLVKQLSSNF GAISSVLNDI LSRLDKPEAE VQIDRLITGR LQSLQTYVTQ QLIRAAEIRA
SANLAATKMS ECVLGQSKRV DFCGKGYHLM SFPQSAPHGV VFLHVTYVPA QEKNFTTAPA
ICHDGKAHFP REGVFVSNGT HWFVTQRNFY EPQIITTDNT FVSGNCDVVI GIVNNTVYDP
LQPELDSFKE ELDKYFKNHT SPDVDLGDIS GINASVVNIQ KEIDRLNEVA KNLNESLIDL
QELGKYEQGS GYIPEAPRDG QAYVRKDGEW VLLSTFLGRS LEVLFQGPGS LPETGGGSDY
KDDDDKGGGG SGGGGSGGGG SGGGGSGGGG SHHHHHH

The human proteins with which the COVID peptides share sequence are shown in Tables 2 to 5, along with alignments to parts of their sequences.

TABLE 2

| | Sequence | | SEQ |
|---|---|---|---|
| Peptide/human protein | location | Sequence | ID NO |
| PA01 | — | MYSFVSEETGTLIVNSV | 1 |
| S-formylglutathione hydrolase | 120-127 | MYSYVTEE | 7 |
| PTPN13 | 1496-1502 | YSFVTEE | 8 |
| protein O-mannosyl-transferase | 744-749 | VSEETG | 9 |
| TMTC1 | | | |
| LRRC8E | 342-352 | YSFRSVREETG | 10 |
| PRKAB2 | 193-200 | MYAFRSEE | 11 |
| ZFP106 | 874-883 | YSFFSEEGTG | 12 |

TABLE 3

| Peptide/ | Sequence | | SEQ |
|---|---|---|---|
| human protein | location | Sequence | ID NO |
| P87 | — | LLHAPATVCGPKKST | 2 |
| KCNC2 | 123-132 | LHCPADVCGP | 13 |
| SHANK2 | 284-292 | LLHEHATVC | 14 |

TABLE 4

| | | | SEQ |
|---|---|---|---|
| Peptide/ | Sequence | | ID |
| human protein | location | Sequence | NO |
| Ps556 | — | NKKF---LPFQQFGRDIA | 3 |
| serpin B4 | 4-19 | LSEANTKFMFDL-FQQF | 15 |
| (isoform 2) | | | |
| KCNB1 | 279-290 | LTESN-KSV--LQFQ | 16 |

TABLE 5

| | Sequence | | SEQ |
|---|---|---|---|
| Peptide/human protein | location | Sequence | ID NO |
| P177 | — | PHGVVFLHVTYVPAQ | 4 |
| HEAT repeat-containing | 172-180 | GVVFPHVSY | 17 |
| protein 5B isoform | | | |
| N-acetylmuramoyl-L-alanine | 406-416 | FLYVHHTYVPA | 18 |
| amidase (isoform 1 precursor) | | | |
| myeloid cell surface antigen | 102-108 | LNV--TYVP | 19 |
| CD33 isoform 2 | | | |

### Example 2—Subcutaneous Immunisation of Mice with COVID Peptide Cocktail Including Different Adjuvants Induces Inflammation of the Heart

Mice were immunised subcutaneously with two doses, 1 week apart, of a cocktail of the four COVID peptides including either no adjuvant (control) or one of the following adjuvants: alum +MPLA, R848 (Resiquimod), or AS03+MPLA.

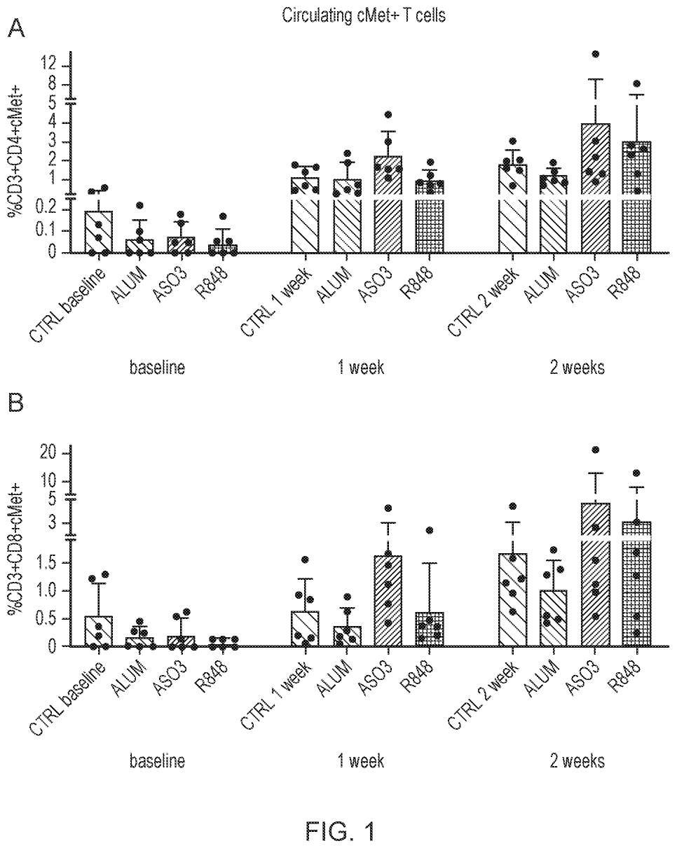

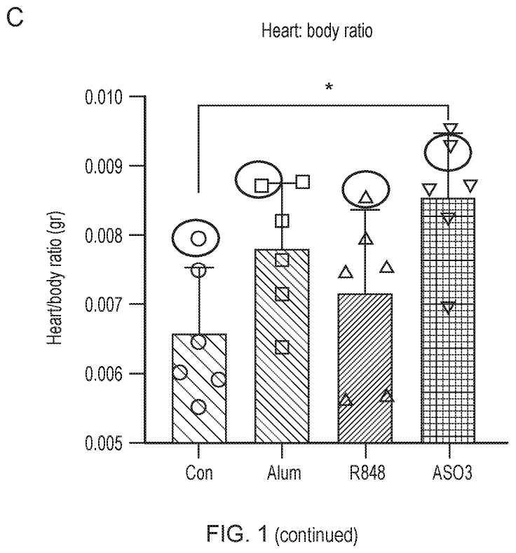

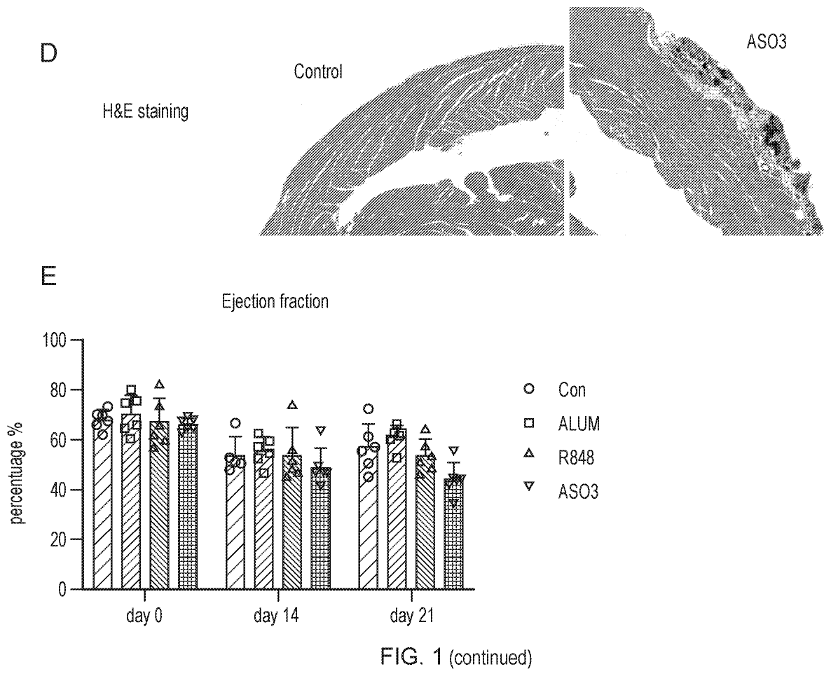

The levels of circulating CD3+CD4+cMet+ T cells (FIG. 1A) and CD3+CD8+cMet+ T cells (FIG. 1B) in the mice were measured. The level of CD3+CD4+cMet+ T cells had increased markedly 1 week after the first immunisation and remained high 1 week after the second immunisation, regardless of whether adjuvant was included in the cocktail (see FIG. 1A). Similarly, the level of CD3+CD8+cMet+ T cells increased by 1 week after the first immunisation and was even higher 1 week after the second immunisation (see FIG. 1B). The highest level of both CD3+CD4+cMet+ and CD3+CD8+cMet+ T cells was observed when AS03+MPLA adjuvant was included in the cocktail.

The heart weight to body weight ratio of the mice was measured (FIG. 1C). A higher heart:body weight ratio indicates a heavier heart, which may be due to inflammation. Mice immunised with peptide cocktail comprising AS03+MPLA adjuvant had a significantly higher heart:body weight ratio than mice treated with a peptide cocktail lacking adjuvant (control), indicating a higher degree of heart inflammation was induced by peptide cocktail comprising AS03+MPLA adjuvant.

FIG. 1D shows haematoxylin and eosin staining of a heart from a control mouse (immunised with peptide cocktail with no adjuvant) and a heart from a mouse immunised with peptide cocktail including AS03+MPLA adjuvant.

Heart ejection fraction (percentage of blood leaving the heart each time it contracts) of the mice was measured (FIG. 1E). Heart ejection fraction was reduced by 14 days, and even further reduced by 21 days, for all mice, indicating that immunisation with the peptide cocktail reduces heart function. The greatest reduction in heart ejection fraction was observed when the peptide cocktail included AS03+MPLA adjuvant.

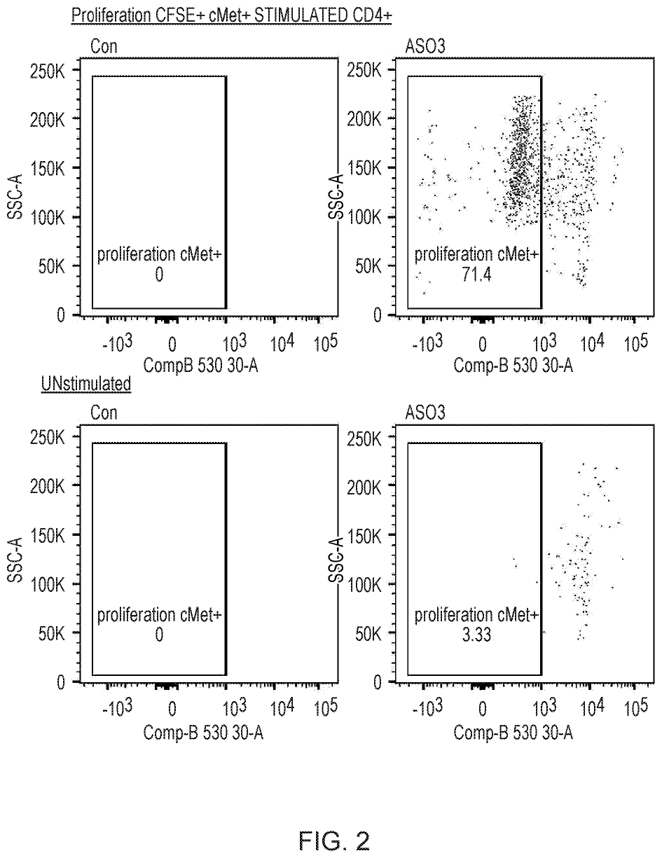

Proliferation of CD4+cMet+ T cells upon immunisation of mice with peptide cocktail, with or without AS03+MPLA adjuvant, was compared to proliferation in non-immunised mice using fluorescence-activated cell sorting (FACS) (FIG. 2). The data show greatly increased proliferation of CD4+cMet+ T cells in mice immunized with peptide cocktail including AS03+MPLA.

In summary, these data indicate that immunisation of mice with a cocktail of the COVID peptides induces inflammation of the heart, correlating with reduced heart function and an increase in circulating cMet+ T cells. The adjuvant AS03+MPLA particularly enhances the effect of administration of the peptide cocktail.

### Example 3—Intranasal Immunisation of Mice with COVID Peptide Cocktail Including AS03 Induces Inflammation of the Heart

To mimic infection with SARS-CoV-2 coronavirus, mice were immunised via the intranasal route with two doses, 1 week apart, of a cocktail of the four COVID peptides and AS03+MPLA adjuvant. Control mice were immunised with AS03+MPLA alone.

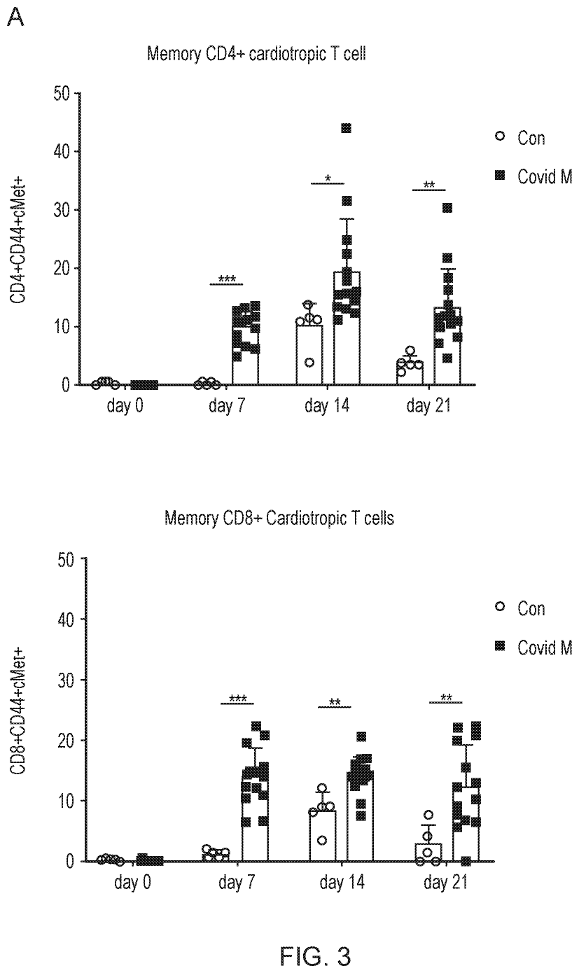

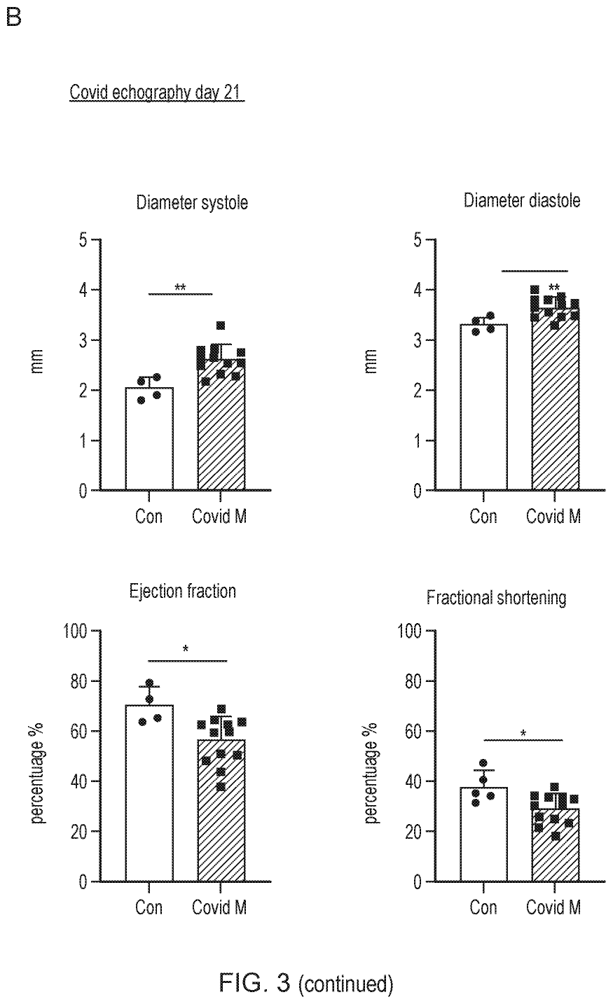

The levels of circulating CD4+cMet+ T cells and CD8+cMet+ T cells in the mice were measured (FIG. 3A). Levels of both types of T cells increased significantly in mice immunised with the peptide cocktail in comparison to control mice over the three weeks after the first immunisation.

Echocardiography was used to measure cardiac function three weeks after the first immunisation (FIG. 3B). Mice immunised with the peptide cocktail had increased heart diameter at systole (contracted heart) and diastole (relaxed heart), reduced ejection fraction (percentage of blood leaving the heart each time it contracts) and reduced fractional shortening (percentage change in left ventricle diameter during systole).

FIG. 3C shows haematoxylin and eosin staining of hearts from two mice immunised with peptide cocktail including AS03+MPLA adjuvant. Both hearts show characteristics of myocarditis.

Together, these data show that immunisation of mice via the intranasal route with a cocktail of the COVID peptides induces inflammation of the heart, correlating with reduced heart function and an increase in circulating cMet+ T cells.

### Example 4—Immunisation of Mice with Individual COVID Peptides Increases cMet+ T Cells

Mice were immunised with two doses, 1 week apart, of a single COVID peptide with AS03+MPLA adjuvant. Control mice were immunised with adjuvant alone.

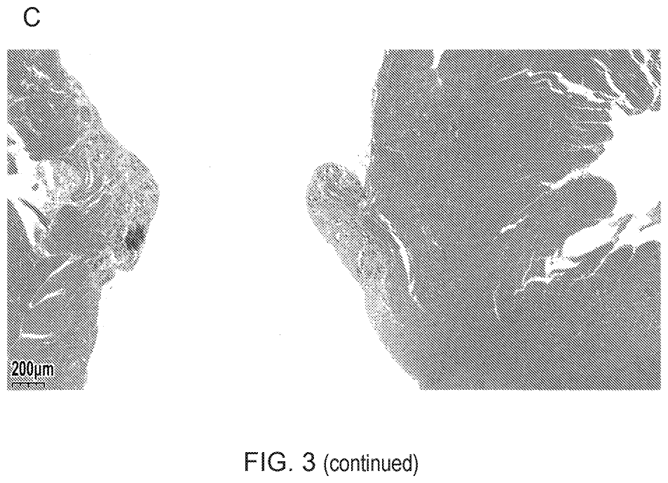

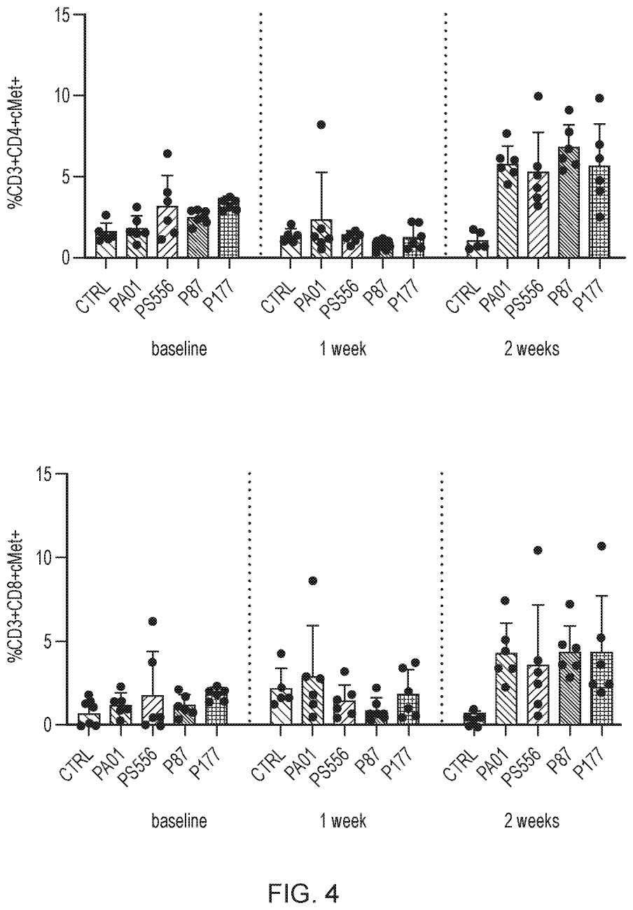

The levels of circulating CD3+CD4+cMet+ T cells and CD3+CD8+cMet+ T cells in the mice were measured (FIG. 4). The levels of both T cell types had increased markedly after two weeks from the first immunisation in mice immunised with any of the COVID peptides, but did not increase in control mice.

These data indicate that each of the identified COVID peptides alone can induce expansion of cardiotropic cMet+ T cells.

### Example 5—Flow Cytometry Results from Acute Myocarditis Patient

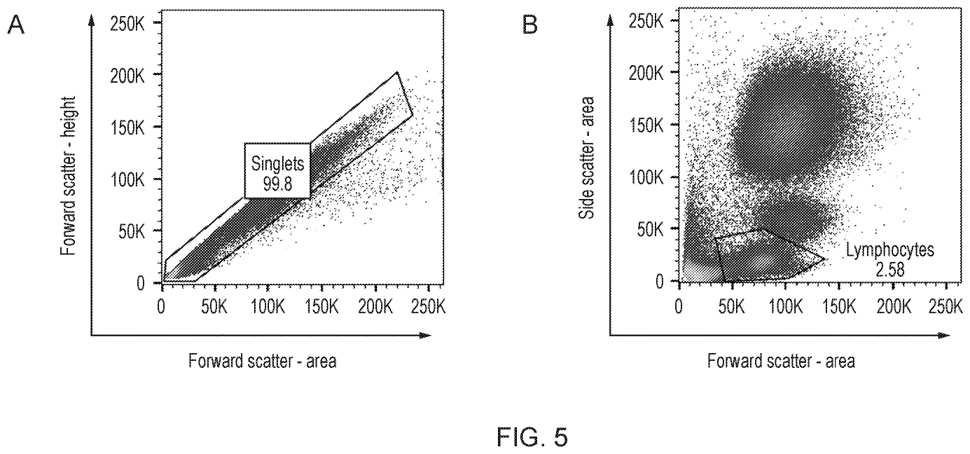

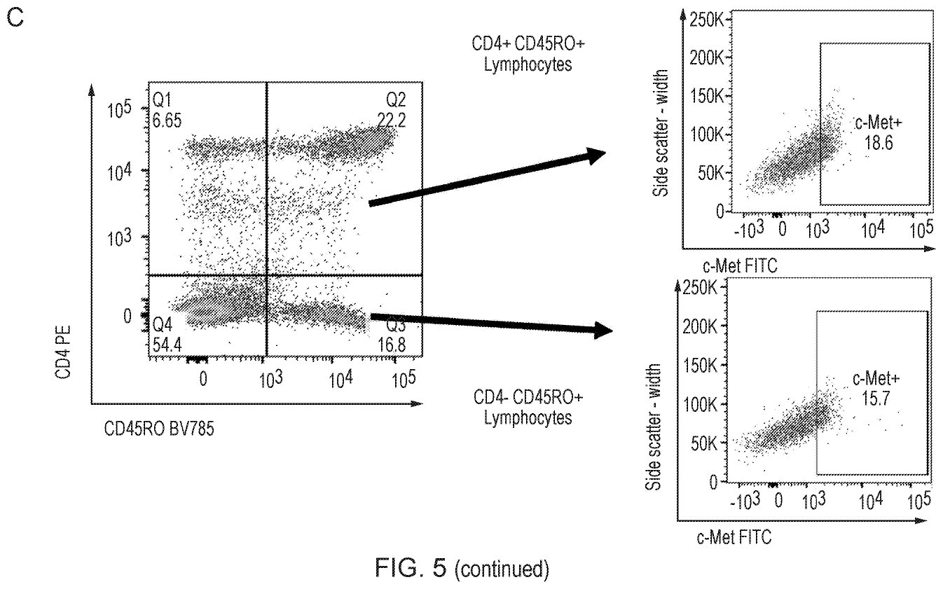

100 μL of whole peripheral blood from a patient with acute myocarditis was stained with anti-CD4, anti-CD45RO and anti-cMet antibodies in 52 minutes and the sample analysed by flow cytometry. FIG. 5 shows the gating strategy from data for single cells (A) and lymphocytes identified within the blood (B). Granulocyte and monocyte populations also seen. With gating on single cell lymphocytes, cMet expression of 15% to 18% is seen in this acute myocarditis patient with both CD4+CD45RO+ T cells, as well as CD4− (presumed to be CD8+) CD45RO+ T cells (C). Similar results were obtained in two other acute myocarditis patients.

1. A peptide selected from the group consisting of:
(SEQ ID NO 1)
MYSFVSEETGTLIVNSV,
(SEQ ID NO 2)
LLHAPATVCGPKKST,
(SEQ ID NO 3)
NKKFLPFQQFGRDIA,
(SEQ ID NO 4)
PHGVVFLHVTYVPAQ,
and variants thereof having at least 80% identity to any one of SEQ ID NOs 1, 2, 3 or 4.

2. A polynucleotide encoding one or more of the peptides as defined in claim 1.

3. A composition comprising one or more of the peptides as defined in claim 1, one or more polynucleotides encoding one or more of the peptides, or combinations thereof.

4-6. (canceled)

7. An antibody or chimeric antigen receptor (CAR) capable of binding to one or more of the peptides as defined in claim 1.

8. A CAR T cell expressing the CAR as defined in claim 7.

9. The CAR T cell of claim 8, wherein the CAR T cell is a CAR regulatory T cell (CAR Treg).

10. A pharmaceutical or immunomodulatory composition comprising:
(a) one or more of the peptides as defined in claim 1;
(b) one or more polynucleotides encoding one or more of the peptides;
(c) one or more vectors encoding one or more of the peptides, or comprising one or more polynucleotides encoding one or more of the peptides;
(d) one or more antibodies or CARs capable of binding to one or more of the peptides;
(e) one or more CAR T cells expressing the CAR capable of binding to one or more of the peptides;
or combinations thereof,
and a pharmaceutically acceptable carrier, vehicle, diluent or excipient.

11. (canceled)

12. A method of modulating an immune response in a human or animal, said method comprising administering to the subject
(a) a composition comprising one or more the peptides as defined in claim 1,
(b) a vector encoding one or more of the peptides, or comprising one or more polynucleotides encoding one or more of the peptides,
(c) an antibody or CAR capable of binding to one or more of the peptides,
(d) a CAR T cell expressing the CAR capable of binding to one or more the peptides,
(e) a pharmaceutical or immunomodulatory composition comprising
(i) one or more antibodies or CARs capable of binding to one or more of the peptides,
(ii) one or more CAR T cells expressing the CAR capable of binding to one or more of the peptides,
and a pharmaceutically acceptable carrier, vehicle, diluent or excipient
or combinations thereof.

13. (canceled)

14. A method of treating or preventing an inflammatory condition or of treating COVID-19 in a subject, said method comprising administering to the subject
(a) an antibody or CAR capable of binding to one or more of the peptides as defined in claim 1,
(b) a CAR T cell expressing the CAR capable of binding to one or more of the peptides,
(c) a pharmaceutical or immunomodulatory composition comprising
(i) one or more antibodies or CARs capable of binding to one or more of the peptides,
(ii) one or more CAR T cells expressing the CAR capable of binding to one or more of the peptides,
and a pharmaceutically acceptable carrier, vehicle, diluent or excipient
or combinations thereof.

15-17. (canceled)

18. The method of claim 14, wherein the inflammatory condition is
(a) a cardiac inflammatory condition,
(b) myocarditis,
(c) or pericarditis.

19. (canceled)

20. A major histocompatibility complex (MHC)-multimer comprising one or more of the peptides as defined in claim 1.

21-22. (canceled)

23. A kit comprising:
(a) a composition comprising one or more of the peptides as defined in claim 1, or one or more polynucleotides encoding one or more of the peptides;
(b) a vector encoding one or more of the peptides, or comprising one or more polynucleotides encoding one or more of the peptides;
(c) an antibody or CAR capable of binding to one or more of the peptides;
(d) a CAR T cell expressing the CAR capable of binding to one or more of the peptides;
(e) a pharmaceutical or immunomodulatory composition comprising
(i) one or more of the peptides;
(ii) one or more polynucleotides encoding one or more of the peptides;
(iii) one or more vectors encoding one or more of the peptides, or comprising one or more polynucleotides encoding one or more of the peptides;
(iv) one or more antibodies or CARs capable of binding to one or more of the peptides;
(v) one or more CAR T cells expressing the CAR capable of binding to one or more of the peptides;
and a pharmaceutically acceptable carrier, vehicle, diluent or excipient;
(f) an MHC-multimer comprising one or more of the peptides;
or combinations thereof.

24. A method for determining in a sample a level of antigen-specific T cells expressing a T cell receptor (TCR) that binds to one or more of the peptides as defined in claim 1, the method comprising the steps of
(a) incubating the sample with one or more of the peptides; and
(b) determining the level of antigen-specific T cells in the sample by detecting binding of the one or more peptide to the TCR of antigen-specific T cells in the sample.

25-29. (canceled)

30. A method for diagnosing a condition in a subject or for predicting whether a subject will develop a condition, the method comprising the steps of:
(a) determining a level of antigen-specific T cells expressing a T cell receptor (TCR) that binds to one or more of the peptides as defined in claim 1 in a sample obtained from the subject by:
(i) incubating the sample with the one or more peptides; and
(ii) determining the level of antigen-specific T cells in the sample by detecting binding of the one or more peptides to the TCR of antigen-specific T cells in the sample; and
(b) comparing the level of antigen-specific T cells in the sample to an antigen-specific T cell reference value,
wherein the level of antigen-specific T cells in the sample compared to the antigen-specific T cell reference value is indicative of the presence or absence of the condition in the subject.

31-32. (canceled)

33. The method of claim 30, wherein the condition is an inflammatory condition, a cardiac inflammatory condition, wherein the cardiac inflammatory condition is myocarditis or pericarditis.

34-36. (canceled)

37. The method of claim 30, wherein the method further comprises the steps of:
(a) determining a level of cMet-positive T cells in a sample obtained from the subject; and
(b) comparing the level of cMet-positive T cells in the sample to a cMet-positive T cell reference value,
wherein the level of cMet-positive T cells in the sample compared to the cMet-positive T cell reference value is indicative of the presence or absence of the condition in the subject or predictive of whether the subject will develop the condition.

38. (canceled)

39. The method of claim 37, wherein the cMet-positive T cells are
(a) cardiotropic T cells or virus-induced cardiotropic T cells;
(b) CCR4-positive T cells;
(c) CXCR3-positive T cells;
(d) CD4-positive T cells;
(e) CD8-positive T cells;
or combinations thereof.

40-43. (canceled)

44. The method of claim 30, wherein the subject is
(a) infected with COVID-19 virus;
(b) suffering from one or more morbidity associated with COVID-19;
(c) suffering from long COVID;
or combinations thereof.

45-46. (canceled)

47. The method of claim 30, wherein the subject
(a) has been vaccinated against COVID-19 or has been vaccinated against COVID-19 within 6 weeks prior to performing said method of diagnosis or prediction;
(b) has been vaccinated against COVID-19 using an mRNA vaccine or has been vaccinated against COVID-19 using BNT162b2 vaccine or mRNA-1273 vaccine;
or combinations thereof.

48-51. (canceled)

52. The method of claim 30, wherein the subject is
(a) a male human;
(b) from 10 to 60 years of age;
or combinations thereof.

53-56. (canceled)
